(19) United States
(12) Patent Application Publication
(10) Pub. No.: US 2007/0287726 A1
(43) Pub. Date: Dec. 13, 2007

(54) 5-Substituted 1H-Pyrrolo [3,2-B] Pyridines

(75) Inventor: Andreas Palmer, Singen (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/658,453

(22) PCT Filed: Jul. 29, 2005
(86) PCT No.: PCT/EP05/53729

§ 371 Date: Feb. 13, 2007

(30) Foreign Application Priority Data
Aug. 2, 2004 (EP) 04103715.1

(51) Int. Cl. A61K 31/437; A61P 1/00; C07D 471/02

(52) U.S. Cl. 514/300; 546/113

(57) ABSTRACT

The invention relates to compounds of the formula (1) 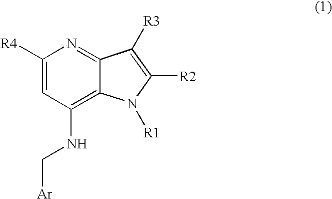 wherein the substituents and symbols have the meanings indicated in the description. The compounds have gastric acid secretion inhibiting and excellent gastric and intestinal protective action properties.

### TECHNICAL FIELD

The invention relates to novel compounds, which are used in the pharmaceutical industry as active compounds for preparing medicaments.

### PRIOR ART

In the international patent application WO 99/28322 heterocyclic compounds are described which are said to be effective as inhibitors of the gastrointestinal H⁺K⁺-ATPase and thereby as inhibitors of gastric acid secretion. Pyrrolo[3,2-b]pyridine derivatives are mentioned among other heterocyclic compounds, but no concrete example for this particular heterocyclic system is described.

In the International patent application WO 00/10999 imidazo[1,2-a]pyridines with a particular substitution pattern are disclosed which compounds can be used in the prevention and treatment of gastrointestinal inflammatory diseases.

### DESCRIPTION OF THE INVENTION

The invention relates to compounds of the formula 1 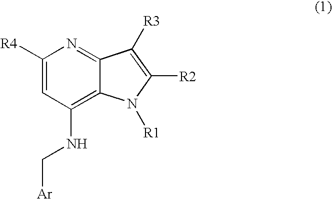
in which
R1 is hydrogen, a radical CH₂—R11 or a radical CH(OR12)-R11
   where
   R11 is hydrogen, 1-4C-alkyl or a group Cy,
   R12 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, fluoro-1-4C-alkyl, 1-4C-alkylcarbonyl or the radical —CO—N(R121)(R122) where
      R121 is hydrogen, 1-7C-alkyl, hydroxy-1-4C-alkyl, 1-4C-alkoxy-1-4C-alkyl or 3-7C-cycloalkyl and
      R122 is hydrogen or 1-7C-alkyl,
      or where
      R121 and R122 together and including the nitrogen atom to which they are attached form a pyrrolidino, piperidino, morpholino, aziridino or azetidino radical,
R2 is hydrogen, 1-4C-alkyl, 3-7C-cycloalkyl, 3-7C-cycloalkyl-1-4C-alkyl, 1-4C-alkoxy-1-4C-alkyl, 14C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32, where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
   where
   R41 is hydrogen, 1-7C-alkyl, hydroxy-1-4C-alkyl or 1-4C-alkoxy-1-4C-alkyl or 3-7C-cycloalkyl and
   R42 is hydrogen, 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R6 is hydrogen, 1-4C-alkyl, 1-4C-alkoxy, 1-4C-alkoxycarbonyl, halogen, trifluoromethyl or hydroxyl,
   R7 is hydrogen, 1-4C-alkyl, 1-4C-alkoxy, hydroxyl or halogen and
   R8 is hydrogen, 1-4C-alkyl or halogen,
where
aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
   and Cy is a tetrahydrofuryl group or a mono- or bicyclic aromatic residue as defined for Ar or aryl,
   and the salts of these compounds.

1-4C-Alkyl denotes straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl denotes cyclopropyl, cyclobutyl, cyclopentyl, cyclohexyl and cycloheptyl, among which cyclopropyl, cyclobutyl and cyclopentyl are preferred.

3-7C-Cycloalkyl-1-4C-alkyl denotes one of the abovementioned 1-4C-alkyl radicals which is substituted by one of the abovementioned 3-7C-cycloalkyl radicals. Examples which may be mentioned are the cyclopropylmethyl, the cyclohexylmethyl and the cyclohexylethyl radicals.

1-4C-Alkoxy denotes radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

1-4C-Alkoxy-1-4C-alkyl denotes one of the abovementioned 1-4C-alkyl radicals which is substituted by one of the abovementioned 1-4C-alkoxy radicals. Examples which may be mentioned are the methoxymethyl, the methoxyethyl and the butoxyethyl radicals.

1-4C-Alkoxycarbonyl (—CO-1-4C-alkoxy) denotes a carbonyl group to which is attached one of the abovementioned 1-4C-alkoxy radicals. Examples which may be mentioned are the methoxycarbonyl (CH₃O—C(O)—) and the ethoxycarbonyl (CH₃CH₂O—C(O)—) radicals.

2-4C-Alkenyl denotes straight-chain or branched alkenyl radicals having 2 to 4 carbon atoms. Examples which may be mentioned are the 2-butenyl, 3-butenyl, 1-propenyl and the 2-propenyl (allyl) radicals.

2-4C-Alkynyl denotes straight-chain or branched alkynyl radicals having 2 to 4 carbon atoms. Examples which may be mentioned are the 2-butynyl, the 3-butynyl and, preferably, the 2-propynyl (propargyl radicals).

Fluoro-1-4C-alkyl represents one of the aforementioned 1-4C-alkyl groups, which is substituted by one or more fluorine atoms. An example which may be mentioned are the trifluoromethyl group, the difluoromethyl, the 2-fluoroethyl, the 2,2-difluoroethyl or the 2,2,2-trifluoroethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl and the 3-hydroxypropyl group. Hydroxy-1-4C-alkyl within the scope of the invention is understood to include 1-4C-alkyl groups with two or more hydroxy groups. Examples which may be mentioned are the 3,4-dihydroxybutyl and in particular the 2,3-dihydroxypropyl group.

Hydroxy-3-4-C-alkenyl denotes abovementioned 3-4-C-alkenyl radicals which are substituted by a hydroxyl group. Examples which may be mentioned are the 1-hydroxypropenyl or the 1-hydroxy-2-butenyl radical.

Hydroxy-3-4-C-alkinyl denotes abovementioned 3-4-C-alkinyl radicals which are substituted by a hydroxyl group. Examples which may be mentioned are the 1-hydroxypropinyl or the 1-hydroxy-2-butinyl radical.

For the purpose of the invention, halogen is bromine, chlorine and fluorine.

1-4C-Alkoxy-1-4C-alkoxy denotes one of the abovementioned 1-4C-alkoxy radicals which is substituted by a further 1-4C-alkoxy radical. Examples which may be mentioned are the radicals 2-(methoxy)ethoxy (CH₃—O—CH₂—CH₂—O—) and 2-(ethoxy)ethoxy(CH₃—CH₂—O—CH₂—CH₂—O—).

1-4C-Alkoxy-1-4C-alkoxy-1-4C-alkyl denotes one of the abovementioned 1-4C-alkoxy-1-4C-alkyl radicals which is substituted by one of the abovementioned 1-4C-alkoxy radicals. An example which may be mentioned is the radical 2-(methoxy)ethoxymethyl(CH₃—O—CH₂—CH₂—O—CH₂—).

1-7C-Alkyl denotes straight-chain or branched alkyl radicals having 1 to 7 carbon atoms. Examples which may be mentioned are the heptyl, isoheptyl-(5-methylhexyl), hexyl, isohexyl-(4-methylpentyl), neohexyl-(3,3-dimethylbutyl), pentyl, isopentyl-(3-methylbutyl), neopentyl-(2,2-dimethylpropyl), butyl, isobutyl, sec-butyl, tert-butyl, propyl, isopropyl, ethyl and methyl radicals.

1-4C-Alkylcarbonyl denotes a radical which, in addition to the carbonyl group, contains one of the abovementioned 1-4C-alkyl radicals. An example which may be mentioned is the acetyl radical.

2-4-C-Alkenylcarbonyl denotes a radical which, in addition to the carbonyl group, contains one of the abovementioned 2-4C-alkenyl radicals. An example which may be mentioned is the ethenylcarbonyl or the 2-propenylcarbonyl radical.

2-4-C-Alkinylcarbonyl denotes a radical which, in addition to the carbonyl group, contains one of the abovementioned 2-4C-alkinyl radicals. An example which may be mentioned is the ethinylcarbonyl or the 2-propinylcarbonyl radical.

2-4C-Alkenyloxy denotes a radical which, in addition to the oxygen atom, contains a 2-4C-alkenyl radical. An example which may be mentioned is the allyloxy radical.

Carboxy-1-4C-alkyl denotes, for example, the carboxymethyl (—CH₂COOH) or the carboxyethyl (—CH₂CH₂COOH) radical.

1-4C-Alkoxycarbonyl-1-4C-alkyl denotes one of the abovementioned 1-4C-alkyl radicals which is substituted by one of the abovementioned 1-4C-alkoxycarbonyl radicals. An example which may be mentioned is the ethoxycarbonylmethyl (CH₃CH₂OC(O)CH₂—) radical.

Aryl-1-4C-alkyl denotes an aryl-substituted 1-4C-alkyl radical. An example which may be mentioned is the benzyl radical.

Aryl-1-4C-alkoxy denotes an aryl-substituted 1-4C-alkoxy radical. An example which may be mentioned is the benzyloxy radical.

Mono- or di-1-4C-alkylamino radicals contain, in addition to the nitrogen atom, one or two of the abovementioned 1-4C-alkyl radicals. Preference is given to di-1-4C-alkylamino and in particular to dimethyl-, diethyl- or diisopropylamino.

Mono- or di-1-4C-alkylamino-1-4C-alkyl denotes one of the abovementioned 1-4C-alkyl radicals which is substituted by one of the abovementioned mono- or di-1-4C-alkylamino radicals. Preferred mono- or di-1-4C-alkylamino-1-4C-alkyl radicals are the mono- or di-1-4C-alkylaminomethyl radicals. An Example which may be mentioned is the dimethylaminomethyl (CH₃)₂N—CH₂ radical.

1-4C-Alkylcarbonylamino denotes an amino group to which a 1-4C-alkylcarbonyl radical is attached. Examples which may be mentioned are the propionylamino (C₃H₇C(O)NH—) and the acetylamino (acetamido, CH₃C(O)NH—) radicals.

1-4C-Alkoxycarbonylamino denotes an amino radical which is substituted by one of the abovementioned 1-4C-alkoxycarbonyl radicals. Examples which may be mentioned are the ethoxycarbonylamino and the methoxycarbonylamino radicals.

1-4C-Alkoxy-1-4C-alkoxycarbonyl denotes a carbonyl group to which one of the abovementioned 1-4C-alkoxy-1-4C-alkoxy radicals is attached. Examples which may be mentioned are the 2-(methoxy)ethoxycarbonyl (CH₃—O—CH₂CH₂—O—CO—) and the 2-(ethoxy)ethoxycarbonyl(CH₃CH₂—O—CH₂CH₂—O—CO—) radicals.

1-4C-Alkoxy-1-4C-alkoxycarbonylamino denotes an amino radical which is substituted by one of the abovementioned 1-4C-alkoxy-1-4C-alkoxycarbonyl radicals. Examples which may be mentioned are the 2-(methoxy)ethoxycarbonylamino and the 2-(ethoxy)ethoxycarbonylamino radicals.

Radicals Ar which may be mentioned are, for example, the following substituents: 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoro methoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.

Suitable salts of compounds of the formula 1 are—depending on the substitution—in particular all acid addition salts. Particular mention may be made of the pharmacologically acceptable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in the salt preparation in an equimolar ratio or in a ratio differing therefrom, depending on whether the acid is a mono- or polybasic acid and on which salt is desired—in an equimolar quantitative ratio or one differing therefrom.

Pharmacologically unacceptable salts, which can be initially obtained, for example, as process products in the preparation of the compounds according to the invention on an industrial scale, are converted into pharmacologically acceptable salts by processes known to the person skilled in the art.

It is known to the person skilled in the art that the compounds according to the invention and their salts can, for example when they are isolated in crystalline form, comprise varying amounts of solvents. The invention therefore also embraces all solvates and, in particular, all hydrates of the compounds of the formula 1, and all solvates and, in particular, all hydrates of the salts of the compounds of the formula 1.

An embodiment (embodiment a) of the invention are compounds of the formula 1, in which R1 is hydrogen and R2, R3, R4, and Ar have the meanings as indicated in the outset.

Another embodiment (embodiment b) of the invention are compounds of the formula 1, in which R1 is a radical CH₂—R11 or a radical CH(OR12)-R11, and R11, R12, R2, R3, R4, and Ar have the meanings as indicated in the outset.

Another embodiment (embodiment c) of the invention are compounds of the formula 1, in which R2 is 1-4C-alkyl and R1, R3, R4, and Ar have the meanings as indicated in the outset.

Another embodiment (embodiment d) of the invention are compounds of the formula 1, in which R2 is hydrogen, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl, and R1, R3, R4, and Ar have the meanings as indicated in the outset.

A preferred embodiment according to the present invention is embodiment c.

A particularly preferred embodiment (embodiment e) according to the present invention are compounds of the formula 1, in which R2 and R3 are each a 1-4C-alkyl radical, in particular R2 and R3 are each a methyl radical, and in which R1, R4, and Ar have the meanings as indicated in the outset.

The invention also relates to compounds of the formula 1, in which
R1 is hydrogen or a radical CH₂—R11
   where
   R11 is hydrogen, 1-4C-alkyl, aryl, tetrahydrofuryl, pyridyl, furyl or thienyl
R2 is hydrogen, 1-4C-alkyl, 3-7C-cycloalkyl, 3-7C-cycloalkyl-1-4C-alkyl, 1-4C-alkoxy-1-4C-alkyl, 14C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
   where
   R41 is hydrogen, 1-7C-alkyl, hydroxy-1-4C-alkyl or 1-4C-alkoxy-1-4C-alkyl or 3-7C-cycloalkyl and
   R42 is hydrogen, 1-7C-alkyl,
   or where
   R41 and R42 together and including the nitrogen atom to which they are attached are a pyrrolidino, piperidino, morpholino, aziridino or azetidino radical,
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
   wherein
   R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   R6 is hydrogen, 1-4C-alkyl, 1-4C-alkoxy, 1-4C-alkoxycarbonyl, halogen, trifluoromethyl or hydroxyl,
   R7 is hydrogen, 1-4C-alkyl, 1-4C-alkoxy, hydroxyl or halogen and
   R8 is hydrogen, 1-4C-alkyl or halogen,
      where
aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
   and the salts of these compounds.

Among the compounds of formula 1, those compounds are to be mentioned, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is hydrogen or 1-4C-alkyl
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
   where
   R31 is hydrogen, 1-7C-alkyl, hydroxy-1-4C-alkyl, 1-4C-alkoxy-1-4C-alkyl, or 3-7C-cycloalkyl, and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6
   where
   R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
   R6 is hydrogen, 1-4C-alkyl or 1-4C-alkoxy,
      or
Ar is selected from the group consisting of 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis-(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.
   and the salts of these compounds.

Compounds of the formula 1, which are also to be mentioned, are those in which
R1 is hydrogen or a radical CH₂—R11
   where
   R11 is hydrogen, 1-4C-alkyl, phenyl, tetrahydrofuryl, pyridyl, furyl or thienyl
R2 is hydrogen or 1-4C-alkyl
R3 is hydrogen, 1-4C-alkyl, halogen, 2-4C-alkenyl, 2-4C-alkynyl, hydroxy-1-4C-alkyl, 1-4C-alkoxycarbonyl, cyanomethyl, amino, mono- or di-1-4C-alkylamino, 1-4C-alkylcarbonylamino, 1-4C-alkoxycarbonylamino, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
Ar is a phenyl group substituted by R5 and R6
   where
   R5 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
   R6 is hydrogen, 1-4C-alkyl or 1-4C-alkoxy,
      or
Ar is selected from the group consisting of 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis-(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoro methylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1,2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl.
   and the salts of these compounds.

Among the compounds of formula 1, those compounds are to be particularly mentioned, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is 1-4C-alkyl
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
   where
   R31 is hydrogen, 1-7C-alkyl, hydroxy-1-4C-alkyl, 1-4C-alkoxy-1-4C-alkyl, or 3-7C-cycloalkyl, and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6
   where
   R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
   R6 is hydrogen, 1-4C-alkyl or 1-4C-alkoxy,
      and the salts of these compounds.

Compounds of formula 1, which are also to be particularly mentioned, are those in which
R1 is hydrogen or a radical CH₂—R11
   where
   R11 is hydrogen, 1-4C-alkyl, phenyl, tetrahydrofuryl, furyl or thienyl
R2 is 1-4C-alkyl
R3 is hydrogen, 1-4C-alkyl, halogen, 2-4C-alkenyl, 2-4C-alkynyl, hydroxy-1-4C-alkyl, 1-4C-alkoxycarbonyl, cyanomethyl, amino, mono- or di-1-4C-alkylamino, 1-4C-alkylcarbonylamino, 1-4C-alkoxycarbonylamino, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl,
   or where
   R31 and R32 together and including the nitrogen atom to which they are attached are a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
Ar is a phenyl group substituted by R5 and R6
   where
   R5 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and,
   R6 is hydrogen, 1-4C-alkyl or 1-4C-alkoxy,
      and the salts of these compounds.

Compounds of the formula 1 which are to be emphasized are those compounds, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 1-4C-alkoxy-1-4C-alkyl, fluoro-1-4C-alkyl,
   where
   aryl is phenyl or substituted phenyl having one substituent from the group consisting of 1-4C-alkyl, 1-4C-alkoxy, halogen, and hydroxyl,
R2 is 1-4C-alkyl
R3 is hydrogen, 1-4C-alkyl, halogen, 2-4C-alkenyl, hydroxy-1-4C-alkyl, 1-4C-alkoxycarbonyl, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6
   where
   R5 is hydrogen, 1-4C-alkyl, hydroxy-1-4C-alkyl and
   R6 is 1-4C-alkyl,
      and the salts of these compounds.

Compounds of the formula 1 which are also to be emphasized are those compounds of the formula 1, in which
R1 is hydrogen or a radical CH₂—R11
   where
   R11 is hydrogen, 1-4C-alkyl, phenyl, tetrahydrofuryl or furyl
R2 is 1-4C-alkyl
R3 is hydrogen, 1-4C-alkyl, halogen, 2-4C-alkenyl, hydroxy-1-4C-alkyl, 1-4C-alkoxycarbonyl, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
Ar is a phenyl group substituted by R5 and R6
   where
   R5 is 1-4C-alkyl and
   R6 is 1-4C-alkyl,
      and the salts of these compounds.

Compounds of the formula 1 which are to be particularly emphasized are those compounds, in which
R1 is hydrogen, a radical CH₂—R11, or a radical CH(OR12)-R11
   where
   R11 is hydrogen, 1-4C-alkyl, phenyl, tetrahydrofuryl, furyl or thienyl
   R12 is hydrogen, 1-4C-alkyl
R2 is 1-4C-alkyl
R3 is hydrogen, 1-4C-alkyl, halogen, 2-4C-alkenyl or hydroxy-1-4C-alkyl,
R4 is carboxyl, 1-4-C-alkoxycarbonyl, halogen or the radical —CO—NR41R42,
   where
   R41 is hydrogen, 1-4C-alkyl, hydroxy-1-4C-alkyl, 3-7C-cycloalkyl and
   R42 is hydrogen, 1-4C-alkyl,
   or where
   R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6
   where
   R5 is hydrogen, 1-4C-alkyl, hydroxy-1-4C-alkyl and
   R6 is 1-4C-alkyl,
      and the salts of these compounds.

Compounds of the formula 1 which are also to be particularly emphasized are those compounds, in which
R1 is hydrogen or a radical CH₂—R11
   where
   R11 is hydrogen, 1-4C-alkyl, phenyl, tetrahydrofuryl or furyl
R2 is 1-4C-alkyl
R3 is hydrogen, 1-4C-alkyl, halogen, 2-4C-alkenyl or hydroxy-1-4C-alkyl, or the radical —CO—NR31R32,
   where
   R31 is 1-7C-alkyl, 1-4C-alkoxy-1-4C-alkyl and
   R32 is hydrogen, 1-7C-alkyl
R4 is carboxyl, 1-4-C-alkoxycarbonyl, halogen or the radical —CO—NR41R42,
   where
   R41 is hydrogen, 1-4C-alkyl, 3-7C-cycloalkyl and
   R42 is hydrogen, 1-4C-alkyl,
   or where
   R41 and R42 together and including the nitrogen atom to which they are attached are a pyrrolidino, piperidino, morpholino, aziridino or azetidino radical,
Ar is a phenyl group substituted by R5 and R6
   where
   R5 is 1-4C-alkyl and
   R6 is 1-4C-alkyl,
      and the salts of these compounds.

Exemplary compounds of the formula 1, which are to be particularly emphasized are those compounds, in which
R1 is hydrogen or a radical CH₂—R11
   where
   R11 is hydrogen or phenyl
R2 is 1-4C-alkyl
R3 is hydrogen, 1-4C-alkyl, halogen or hydroxy-1-4C-alkyl,
R4 is carboxyl, 1-4C-alkoxycarbonyl, halogen or the radical —CO—NR41R42,
   where
   R41 is 1-4C-alkyl and
   R42 is hydrogen or 1-4C-alkyl,
Ar is a phenyl group substituted by R5 and R6
   where
   R5 is 1-4C-alkyl and
   R6 is 1-4C-alkyl,
      and the salts of these compounds.

Exemplary compounds of the formula 1, which are to be particularly emphasized are those compounds, in which
R1 is hydrogen
R2 is 1-4C-alkyl
R3 is hydrogen, halogen or hydroxy-1-4C-alkyl,
R4 is carboxyl, halogen or the radical —CO—NR41R42,
   where
   R41 is 1-4C-alkyl and
   R42 is 1-4C-alkyl,
Ar is a phenyl group substituted by R5 and R⁶
   where
   R⁵ is 1-4C-alkyl and
   R⁶ is 1-4C-alkyl,
      and the salts of these compounds.

Among the compounds of the formula 1, the compounds of the formula 1-1 are preferred 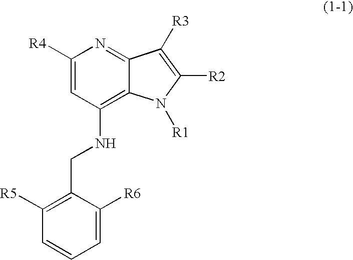
in which
R1 is hydrogen, a radical CH₂—R11 or a radical CH(OR12)-R11
   where
   R11 is hydrogen, 1-4C-alkyl or a group Cy,
   R12 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, fluoro-1-4C-alkyl, 1-4C-alkylcarbonyl or the radical —CO—N(R121)(R122) where
      R121 is hydrogen, 1-7C-alkyl, hydroxy-1-4C-alkyl, 1-4C-alkoxy-1-4C-alkyl or 3-7C-cycloalkyl and
      R122 is hydrogen or 1-7C-alkyl,
      or where
      R121 and R122 together and including the nitrogen atom to which they are attached form a pyrrolidino, piperidino, morpholino, aziridino or azetidino radical,
R2 is hydrogen, 1-4C-alkyl, 3-7C-cycloalkyl, 3-7C-cycloalkyl-1-4C-alkyl, 1-4C-alkoxy-1-4C-alkyl, 14C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-14C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32,
   where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
   where
   R41 is hydrogen, 1-7C-alkyl, hydroxy-1-4C-alkyl or 1-4C-alkoxy-1-4C-alkyl or 3-7C-cycloalkyl and
   R42 is hydrogen, 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxyl,
   where
aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
   and Cy is a tetrahydrofuryl group or a mono- or bicyclic aromatic residue as defined for Ar or aryl,
   and the salts of these compounds.

Also preferred are the compounds of the formula 1-1, in which
R1 is hydrogen or a radical CH₂—R11
   where
   R11 is hydrogen, 1-4C-alkyl, aryl, tetrahydrofuryl, pyridyl, furyl or thienyl
R2 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32, where
   R31 is hydrogen, 1-7C-alkyl, hydroxy-1-4C-alkyl, 1-4C-alkoxy-1-4C-alkyl or 3-7C-cycloalkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
   where
   R41 is hydrogen, 1-7C-alkyl, hydroxy-1-4C-alkyl or 1-4C-alkoxy-1-4C-alkyl or 3-7C-cycloalkyl and
   R42 is hydrogen, 1-7C-alkyl,
   or where
   R41 and R42 together and including the nitrogen atom to which they are attached are a pyrrolidino, piperidino, morpholino, aziridino or azetidino radical,
R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxyl,
   where
aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
   and the salts of these compounds.

Compounds of the formula 1-1, which are to be mentioned, are those, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is hydrogen or 1-4C-alkyl
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
   where
   R31 is hydrogen, 1-7C-alkyl, hydroxy-1-4C-alkyl, 1-4C-alkoxy-1-4C-alkyl, or 3-7C-cycloalkyl, and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
   where

Compounds of the formula 1-1, which are to be particularly mentioned, are those, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 3-7C-cycloalkyl, 3-7C-cycloalkyl-1-4C-alkyl, 1-4C-alkoxy-1-4C-alkyl, fluoro-1-4C-alkyl,
   where
   aryl is phenyl or substituted phenyl having one, two or three identical or different substituents from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is 1-4C-alkyl
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
   where
   R31 is hydrogen, 1-7C-alkyl, hydroxy-1-4C-alkyl, 1-4C-alkoxy-1-4C-alkyl, or 3-7C-cycloalkyl, and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
   and the salts of these compounds.

Compounds of the formula 1-1 which are also to be particularly mentioned, are those, in which
R1 is hydrogen or a radical CH₂—R11
   where
   R11 is hydrogen, 1-4C-alkyl, phenyl, tetrahydrofuryl, furyl or thienyl
R2 is 1-4C-alkyl
R3 is hydrogen, 1-4C-alkyl, halogen, 2-4C-alkenyl, 2-4C-alkynyl, hydroxy-1-4C-alkyl, 1-4C-alkoxycarbonyl, cyanomethyl, amino, mono- or di-1-4C-alkylamino, 1-4C-alkylcarbonylamino, 1-4C-alkoxycarbonylamino, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl,
   or where
   R31 and R32 together and including the nitrogen atom to which they are attached are a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl, 1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
R5 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
   and the salts of these compounds.

Compounds of the formula 1-1, which are to be emphasized, are those, in which
R1 is hydrogen, a radical CH₂—R11, or a radical CH(OR12)-R11
   where
   R11 is hydrogen, 1-4C-alkyl, aryl, tetrahydrofuryl, pyridyl, furyl or thienyl
   R12 is hydrogen, 1-4C-alkyl, 1-4C-alkoxy-1-4C-alkyl, fluoro-1-4C-alkyl,
   where
   aryl is phenyl or substituted phenyl having one substituent from the group consisting of 1-4C-alkyl, 1-4C-alkoxy, halogen, and hydroxyl,
R2 is 1-4C-alkyl
R3 is hydrogen, 1-4C-alkyl, halogen, 2-4C-alkenyl, hydroxy-1-4C-alkyl, 1-4C-alkoxycarbonyl, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
   and the salts of these compounds.

Compounds of the formula 1-1 which are also to mentioned are those, in which
R1 is hydrogen or a radical CH₂—R11
   where
   R11 is hydrogen, 1-4C-alkyl, phenyl, tetrahydrofuryl, pyridyl, furyl or thienyl
R2 is hydrogen or 1-4C-alkyl
R3 is hydrogen, 1-4C-alkyl, halogen, 2-4C-alkenyl, 2-4C-alkynyl, hydroxy-1-4C-alkyl, 1-4C-alkoxycarbonyl, cyanomethyl, amino, mono- or di-1-4C-alkylamino, 1-4C-alkylcarbonylamino, 1-4C-alkoxycarbonylamino, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl,
   or where
   R31 and R32 together and including the nitrogen atom to which they are attached form a pyrrolidino, piperidino, morpholino, aziridino or azetidino radical.
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
R5 is hydrogen, 1-4C-alkyl, hydroxy-1-4C-alkyl, 1-4C-alkoxy, 1-4C-alkoxycarbonyl, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
   and the salts of these compounds.
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl and
R6 is 1-4C-alkyl,
   and the salts of these compounds.

Compounds of the formula 1-1 which are also to be emphasized, are those, in which
R1 is hydrogen or a radical CH₂—R11
   where
   R11 is hydrogen, 1-4C-alkyl, phenyl, tetrahydrofuryl or furyl
R2 is 1-4C-alkyl
R3 is hydrogen, 1-4C-alkyl, halogen, 2-4C-alkenyl, hydroxy-1-4C-alkyl, 1-4C-alkoxycarbonyl, carboxyl, or the radical —CO—NR31R32,
   where
   R31 is hydrogen, 1-7C-alkyl, hydroxy-1-4C-alkyl, 1-4C-alkoxy-1-4C-alkyl and
   R32 is hydrogen, 1-7C-alkyl
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
   where
   R41 is hydrogen, 1-7C-alkyl, hydroxy-1-4C-alkyl-1-4C-alkoxy-1-4C-alkyl, or 3-7C-cycloalkyl and
   R42 is hydrogen or 1-7C-alkyl,
   or where
   R41 and R42 together and including the nitrogen atom to which they are attached are a pyrrolidino, piperidino, morpholino, aziridino or azetidino radical,
R5 is 1-4C-alkyl and
R6 is 1-4C-alkyl,
   and the salts of these compounds.

Compounds of the formula 1-1 which are to be particularly emphasized are those compounds, in which
R1 is hydrogen, a radical CH₂—R11, or a radical CH(OR12)-R11
   where
   R11 is hydrogen, 1-4C-alkyl, phenyl, tetrahydrofuryl, furyl or thienyl
   R12 is hydrogen, 1-4C-alkyl
R2 is 1-4C-alkyl
R3 is hydrogen, 1-4C-alkyl, halogen, 2-4C-alkenyl or hydroxy-1-4C-alkyl,
R4 is carboxyl, 1-4-C-alkoxycarbonyl, halogen or the radical —CO—NR41R42,
   where
   R41 is hydrogen, 1-4C-alkyl, hydroxy-1-4C-alkyl, 3-7C-cycloalkyl and
   R42 is hydrogen, 1-4C-alkyl,
   or where
   R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl and
R6 is 1-4C-alkyl,
   and the salts of these compounds.

Compounds which are also to be particularly emphasized are those compounds, in which
R1 is hydrogen or a radical CH₂—R11
   where
   R11 is hydrogen, 1-4C-alkyl, phenyl, tetrahydrofuryl or furyl
R2 is 1-4C-alkyl
R3 is hydrogen, 1-4C-alkyl, halogen, 2-4C-alkenyl or hydroxy-1-4C-alkyl, or the radical —CO—NR31R32,
   where
   R31 is 1-7C-alkyl, 1-4C-alkoxy-1-4C-alkyl and
   R32 is hydrogen, 1-7C-alkyl
R4 is carboxyl, 1-4-C-alkoxycarbonyl, halogen or the radical —CO—NR41R42,
   where
   R41 is hydrogen, 1-4C-alkyl, 3-7C-cycloalkyl and
   R42 is hydrogen, 1-4C-alkyl,
   or where
   R41 and R42 together and including the nitrogen atom to which they are attached are a pyrrolidino, piperidino, morpholino, aziridino or azetidino radical,
R5 is 1-4C-alkyl and
R6 is 1-4C-alkyl,
   and the salts of these compounds.

Exemplary compounds of the formula 1-1 which are to be particularly emphasized are those compounds, in which
R1 is hydrogen or a radical CH₂—R11
   where
   R11 is hydrogen or phenyl
R2 is 1-4C-alkyl
R3 is hydrogen, 1-4C-alkyl, halogen or hydroxy-1-4C-alkyl,
R4 is carboxyl, 1-4C-alkoxycarbonyl, halogen or the radical —CO—NR41R42,
   where
   R41 is 1-4C-alkyl and
   R42 is hydrogen or 1-4C-alkyl,
R5 is 1-4C-alkyl and
R6 is 1-4C-alkyl,
   and the salts of these compounds.

Exemplary compounds of the formula 1-1 which are also to be particularly emphasized are those compounds, in which
R1 is hydrogen
R2 is 1-4C-alkyl
R3 is hydrogen, halogen or hydroxy-1-4C-alkyl,
R4 is carboxyl, halogen or the radical —CO—NR41R42,
   where
   R41 is 1-4C-alkyl and
   R42 is 1-4C-alkyl,
R5 is 1-4C-alkyl and
R6 is 1-4C-alkyl,
   and the salts of these compounds.

Particularly preferred are the compounds given as final products of formula 1 in the examples, and the salts of these compounds.

The compounds according to the invention can be synthesized from corresponding starting compounds, for example according to the reaction schemes given below. The synthesis is carried out in a manner known to the expert, for example as described in more detail in the following examples.

According to the invention, the compounds of the formula 1 can be prepared as outlined in the reaction schemes 1, 2, 3 and 4, which illustrate processes known to the expert and which use known starting materials.

Compounds of the formula 2 can be transformed into compounds of the formula 1 for example as depicted in scheme 1. Compounds of the formula 2 contain a chlorine atom, which can be substituted versus residues R4, for example by nucleophilic aromatic substitution (for example using water and alcohols) or by palladium-catalysed cross-coupling reactions (for example Heck, Stille, and Sonogashira coupling). Residues R3 can then be introduced for example by treatment of compounds of the formula 3 with electrophiles. Examples that might be mentioned are formylation reactions, using for example the Vilsmeier reagent, halogenation reactions, employing for example N-bromo succinimide, nitrosation reactions, using for example t-butyl nitrite, or acylation reactions, employing for example acetic anhydride. Residues R3 that have been introduced by electrophilic aromatic substitution reactions can then be modified to furnish other residues R3. This might be illustrated by the following examples: Compounds of the formula 4 with R3=halogen are useful starting materials for Palladium-catalysed cross-coupling reactions. Derivatives of the formula 4 with R3=formyl can be reduced to the corresponding alcohols (followed, if desired, by etherification) or oxidized to the corresponding carboxylic acid (followed, if desired, for example by amide or ester formation). Likewise, derivatives of compounds of the formula 4 with R3=carbonyl can be pre-pared by treatment of compounds with R3=formyl with carbon nucleophiles, for example Grignard reagents, followed by oxidation of the intermediate secondary alcohol using a suitable reagent, like e.g. manganese dioxide. 4-Azaindoles of the formula 4 with R3=nitrosyl can be reduced to the corresponding derivatives with R3=amino (followed, if desired, by N-alkylation or acylation). Finally, residues R1 can be introduced, for example by treatment of compounds of the formula 4 with alkylation agents, like for example methyl iodide, benzyl bromide, or epoxides. Residues R1 different from hydrogen can be introduced at any point in the synthesis (for example at the stage of compounds of the formulae 2, 3, or 4). Likewise, conversion of a residue R4 into another residue R4 (for example conversion of an ester into a carboxylic acid or an amide) can be performed at the stage of compounds of the formulae 3, 4, 6, or 1. In the same manner, transformation of residues R3 into other residues R3 can be accomplished not only at the stage of compound 4 but also at the stage of compound 1. The best strategy for the introduction/modification of the residues R1, R3, and R4 depends on the properties of these residues and is obvious for the person skilled in art. It has to be mentioned that—depending on the nature of the residues R1, R2, and R4, compounds of the formulae 2, 3, 4, 5 and 6 might represent specific examples of compounds of the formula 1.

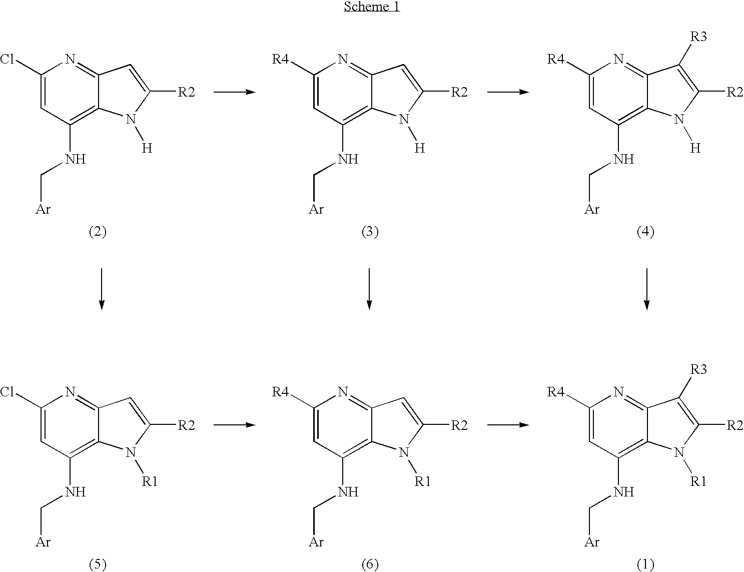

Compounds of the formula 2 can be obtained for example following the synthesis shown in scheme 2 using 4-amino-2,6-dichloro-3-nitropyridine as starting material. Various aromatic residues of the general type CH₂—Ar can be introduced, for example by treatment of 4-amino-2,6-dichloro-3-nitropyridine (7) with suitable electrophiles, like for example benzyl chlorides optionally substituted at the aromatic ring. Further functionalization of the obtained compounds of the formula 8 can then be accomplished for example by palladium-catalysed cross-coupling reactions, for example Stille reaction, using acetylene derivatives bearing different substituents R2. Compounds of the formula 9 contain a nitro function which can be reduced using standard methods, for example acid-catalysed reduction in the presence of tin(II) chloride. Finally, the highly substituted aminopyridines of the formula 10 can be transformed into 4-azaindoles of the formula 2, using a suitable catalyst, for example copper(I) iodide.

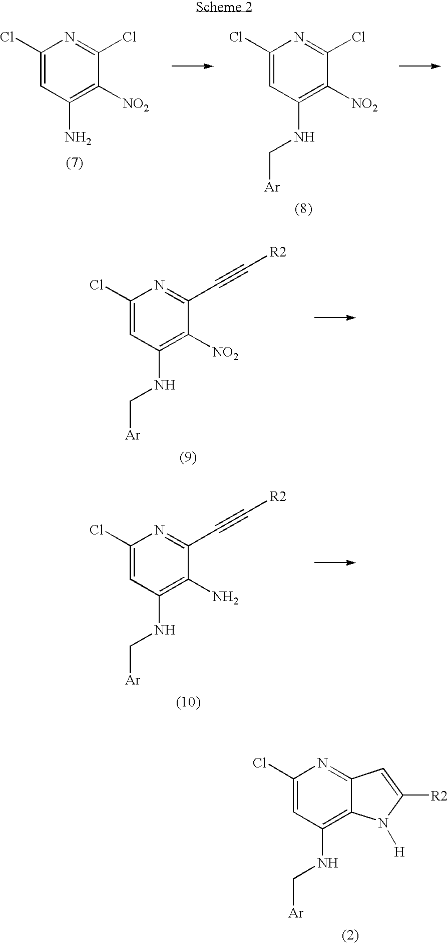

4-Amino-2,6-dichloro-3-nitropyridine is a known compound that is commercially available or can be pre-pared in a manner known to the person skilled in art, for example following the synthesis outlined in J. Heterocycl. Chem. 1965, 2, 196-201, using commercially available 2-Chloro-4-nitropyridine-1-oxide or 2,6-Dichloropyridine as starting material (Scheme 3).

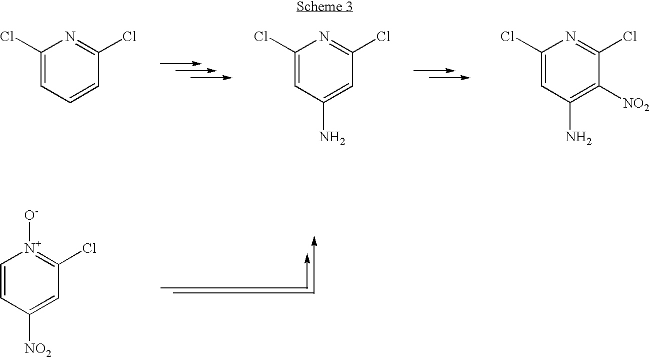

Another approach to 1H-pyrrolo[3,2-b]pyridines of the formula 2 relies on the reductive amination of intermediates of the formula 13 (Scheme 4). The reductive amination reaction comprises (a) the condensation of 1-pyrrolo[3,2-b]pyridines of the formula 13 with aromatic aldehydes in the presence of suitable catalysts, like e.g. acetic acid, and (b) the reduction of the imino derivative obtained in step (a) using a suitable reducing agent, like e.g. sodium triacetoxyborohydride, formic acid, or molecular hydrogen in the presence of palladium on charcoal. Suitable reaction conditions can be identified by the person skilled in art.

Starting materials of the general formula 13 can be prepared using the methods described above: Compounds of the formula 11 can be obtained from 4-amino-2,6-dichloro-3-nitropyridine, for example by palladium-catalysed cross-coupling reactions, for example Stille reaction, using acetylene derivatives bearing different substituents R2. Compounds of the formula 11 contain a nitro function which can be reduced using standard methods, for example acid-catalysed reduction in the presence of tin(II) chloride. Finally, the highly substituted aminopyridines of the formula 12 can be transformed into 4-azaindoles of the formula 13, using a suitable catalyst, for example copper(I) iodide.

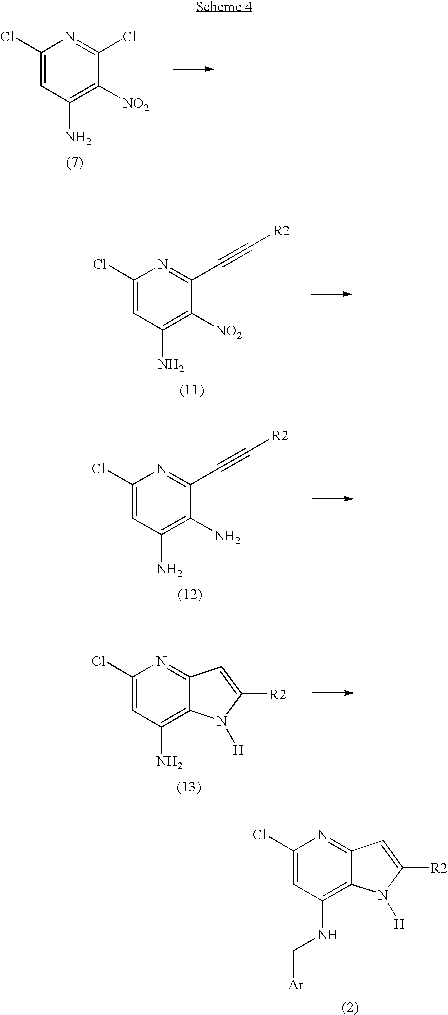

The reaction steps outlined above are carried out in manner known per se, for example as described in more detail in the examples.

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation v stands for volume. For the assignment of NMR (nuclear magnetic resonance) signals, the following abbreviations are used: s (singlet), d (duplet), t (triplet), q (quartet), m_{c} (multiplet centred), b (broad). The following units are used: ml (millilitre), l (litre), mg (milligramme), g (gramme), mmol (millimol), N (normal), M (molar), mbar (millibar), MHz (megahertz).

The following abbreviations are used:

DMF N,N-Dimethylformamide

DMSO Dimethyl sulfoxide

HPLC High pressure liquid chromatography

TBTU O-Benzotriazol-1-yl-N,N,N′,N′-tetramethyluronium tetrafluoroborate

THF Tetrahydrofuran

If melting points were determined after crystallization of the compound, the solvent/solvent mixture that had been used for the purification is given in parentheses. If NMR (nuclear magnetic resonance) chemical shifts are given without integration, overlay of the signal of the corresponding proton of the compound with signals of the solvent, water, or impurities was observed.

### EXAMPLES

### I. Final Products

### 1. (5-Chloro-2-methyl-1H-pyrrolo[3,2-b]pyridin-7-yl)-(2-ethyl-6-methyl-benzyl)-amine

In a flame-dried flask filled with argon, 6-chloro-N⁴-(2-ethyl-6-methyl-benzyl)-2-prop-1-ynyl-pyridine-3,4-diamine (example E, 10.0 g, 32 mmol) was dissolved in dry DMF (80 ml) which had been degassed with argon. Copper(I) iodide (1.20 g, 6.3 mmol) was added and the reaction mixture was heated to 80° C. using an oil-bath which had been pre-heated to this temperature. After a reaction time of 1 hour, the hot dark-brown reaction mixture was poured onto a mixture of a 0.1 M sodium sulfite solution (160 ml) and ice (100 g). A beige suspension was obtained which was stirred for 1 hour at 0° C. The precipitate was removed by filtration, washed with 100 ml portions of water and methanol/water=1:4 (v/v), and dried in vacuo. This afforded 10.7 g of the title compound. The beige solid was pure by means of ¹H-NMR spectroscopy and contained approximately 15% of inorganic salts (as determined by elemental analysis, 91% yield).

Elemental analysis: calculated for C₁₈H20N₃Cl: C: 68.89; H, 6.42; N: 13.39; found: C, 59.63; H, 5.86; N: 11.30.

Melting point: 296° C. (methanol)

¹H-NMR (dmso-d₆, 200 MHz): δ=1,17 (t, 3H), 2.33, 2.35 (2 s, 6H), 2.68 (q, 2H), 4.31 (d, 2H), 6.08 (bs, 1H), 6.18 (bt, 1H), 6.43 (bs, 1H), 7.20 (m_{c}, 3H), 10.80 (bs, 1H).

### 2. Ethyl[7-(2-ethyl-6-methyl-benzylamino)-2-methyl-1H-pyrrolo[3,2-b]pyridine-5-carboxylate]

A solution of (5-chloro-2-methyl-1H-pyrrolo[3,2-b]pyridin-7-yl)-(2-ethyl-6-methyl-benzyl)-amine (example 1, 10.5 g containing approximately 15 weight-% of inorganic salts, 28 mmol) in ethanol (800 ml) and DMF (200 ml) was treated with palladium(II) acetate (1.13 g, 5.0 mmol), 1,3-bis(diphenylphosphano)propane (2.35 g, 5.7 mmol), and potassium carbonate (6.9 g, 5.0 mmol). The reaction mixture was transferred into a 2 l autoclave and a carbon monoxide pressure of 15 bar was applied. The reaction mixture was heated until—after a period of 1.5 hours—a temperature of 190° C. and a carbon monoxide pressure of 30 bar was reached. It was kept for 3 hours at this temperature and was then cooled to room temperature over a period of 1 hour. The pressure was released, the reaction mixture was concentrated until most of the ethanol had been removed, and was then diluted with water (600 ml) and dichloromethane (600 ml). The phases were separated and the aqueous phase was extracted with dichloromethane (2×100 ml). The organic phases were dried over sodium sulfate and concentrated under reduced pressure. The crude product was purified by flash chromatography [300 g of silica gel, eluant:ethyl acetate, then ethyl acetate/methanol=5:1 (v/v)]. The title compound (8.75 g) was isolated as a pale brown, sticky solid which contained approximately 20 weight-% of DMF (as judged from the corresponding ¹H-NMR spectrum). A suspension of the solid in diethyl ether (100 ml) and methanol (10 ml) was stirred for 30 minutes at room temperature. After removal of the solvent and drying in vacuo, a beige solid (6.8 g, 68% yield) was obtained; the pure title compound as judged from the ¹H-NMR spectrum.

Melting point: 274-275° C. (diethyl ether/methanol)

¹H-NMR (dmso-d₆, 200 MHz): δ=1.17 (t, 3H), 1.34 (t, 3H), 2.36, 2.37 (2 s, 6H), 2.70 (q, 2H), 4.32, 4.38 (q, d, 4H), 6.07 (bt, 1H), 6.23 (s, 1H), 7.20 (m_{c}, 4H), 10.94 (s, 1H).

### 3. 7-(2-Ethyl-6-methyl-benzylamino)-2-methyl-1 pyrrolo[3,2-b]pyridine-5-carboxylic acid hydrochloride

Ethyl[7-(2-ethyl-6-methyl-benzylamino)-2-methyl-1H-pyrrolo[3,2-b]pyridine-5-carboxylate] (example 2, 1.55 g, 4.4 mmol) was dissolved in methanol (30 ml) and water (3 ml). Crushed potassium hydroxide (493 mg, 8.80 mmol) was added and the reaction mixture was stirred for 1 hour at 50° C. Another 250 mg (4.46 mmol) of crushed potassium hydroxide was added and the reaction was continued for an additional hour at 50 Cc. The reaction mixture was concentrated under reduced pressure and the residue was treated with water (20 ml) and 2 N hydrochloric acid until a pH-value of 6 was obtained. After addition of methanol (20 ml) a colourless precipitate was formed. The pH-value was re-adjusted to 6 and the suspension was stirred for 1 hour at 0° C. The solid was isolated by filtration and dried in vacuo yielding 1.26 g of pure title compound (79% yield).

Melting point: 325° C. (methanol/water)

¹H-NMR (dmso-d₆, 200 MHz): δ=1.17 (t, 3H), 2.37, 2.43 (2 s, 6H), 2.72 (q, 2H), 4.60 (d, 2H), 6.36 (s, 1H), 7.22 (m_{c}, 4H), 7.71 (bt, 1H), 11.94 (s, 1H), 2 exchangeable protons not visible.

Elemental analysis: calculated for C₁₉H₂₂ClN₃O₂: C, 63.42; H, 6.16; N: 11.68; found: C, 64.23; H, 6.19; N: 11.38.

### 4. 7-(2-Ethyl-6-methyl-benzylamino)-2-methyl-1H-pyrrolo[3,2-b]pyridine-5-carboxylic acid dimethylamide

In a flask filled with argon, a suspension of 7-(2-ethyl-6-methyl-benzylamino)-2-methyl-1H-pyrrolo[3,2-b]pyridine-5-carboxylic acid hydrochloride (example 3, 1.00 g, 2.8 mmol) in dichloromethane (100 ml) and DMF (25 ml) was treated with TBTU (1.10 g, 3.4 mmol). The yellow reaction mixture was refluxed for 1 hour and dimethylamine (1.90 ml of a 2 M solution in THF, 3.8 ml) was added. The reaction was continued for 1 hour at room temperature and the yellow solution was extracted with water (2×50 ml). The aqueous phase was extracted with dichloromethane (20 ml), the combined organic phases were dried over sodium sulfate and concentrated under reduced pressure. A solid residue was obtained which was washed with ethyl acetate (20 ml). In order to remove benzotriazole impurities, the solid was suspended in dichloromethane (50 ml) and water (50 ml) and a pH-value of 10 was adjusted by addition of 2 N sodium hydroxide solution. After addition of methanol (5 ml) two clear phases were obtained which were separated. The aqueous phase was extracted with dichloromethane (3×50 ml). The combined organic phases were dried over sodium sulfate and the solvent was removed in vacuo. The title compound (740 mg, 76% yield) was obtained as a beige solid, pure by means of ¹H-NMR spectroscopy.

¹H-NMR (dmso-d₆, 200 MHz): δ=1.18 (t, 3H), 2.35, 2.36 (2 s, 6H), 2.70 (q, 2H), 3.00, 3.01 (2 s, 6H), 4.32 (d, 2H), 5.95 (bt, 1H), 6.12 (s, 1H), 6.55 (s, 1H), 7.20 (m_{c}, 3H), 10.74 (s, 1H).

### 5. 3-Bromo-7-(2-ethyl-6-methyl-benzylamino)-2-methyl-1H-pyrrolo[3,2-b]pyridine-5-carboxylic acid dimethylamide

In a flask filled with argon, 7-(2-ethyl-6-methyl-benzylamino)-2-methyl-1H-pyrrolo[3,2-b]pyridine-5-carboxylic acid dimethylamide (example 4, 150 mg, 0.43 mmol) was dissolved in dry DMF (10 ml). The solution was cooled to 0° C., N-bromosuccinimide (84 mg, 0.47 mmol, dissolved in 2 ml of DMF) was added over a period of 10 minutes, and the reaction was continued for 30 minutes at 0° C. Dichloromethane (30 ml) and saturated sodium carbonate solution (30 ml) were added to the reaction mixture, the phases were separated, and the aqueous phase was extracted with dichloromethane (2×10 ml). The combined organic phases were dried over sodium sulfate, and concentrated in vacuo. The crude product was purified by flash chromatography [30 g of silica gel, eluant:ethyl acetate/petrol ether=8:2 (v/v), then ethyl acetate/methanol=10:1 (v/v)] and treatment with a mixture of ethyl acetate (2 ml) and diethyl ether (5 ml). After the suspension had been stirred for 15 minutes, the title compound was removed by filtration and dried in vacuo (150 mg, 81% yield).

Melting point: 307° C. (ethyl acetate/diethyl ether)

¹H-NMR (dmso-d₆, 200 MHz): δ=1.17 (t, 3H), 2.35 (s, 6H), 2.69 (q, 2H), 3.02 (s, 6H), 4.34 (d, 2H), 6.07 (bt, 1H), 6.63 (s, 1H), 7.20 (m_{c}, 3H), 11.17 (s, 1H).

### 6. 7-(2-Ethyl-6-methyl-benzylamino)-3-hydroxymethyl-2-methyl-1H-pyrrolo[3,2-b]pyridine-5-carboxylic acid dimethylamide

In a flask filled with argon, sodium borohydride (70 mg, 1.85 mmol) was added to a suspension of 7-(2-ethyl-6-methyl-benzylamino)-3-formyl-2-methyl-1H-pyrrolo[3,2-b]pyridine-5-carboxylic acid dimethylamide (example F, 1.40 g, 3.7 mmol) in dry ethanol (120 ml). The reaction mixture was stirred for 30 minutes at room temperature and was then treated with another portion of sodium borohydride (70 mg, 1.85 mmol). Stirring was continued for another 1 hour at room temperature. The solution was concentrated to a volume of 60 ml. Dichloromethane (100 ml) and saturated ammonium chloride solution (80 ml) was added. The phases were separated, and the aqueous phase was extracted with dichloromethane (2×40 ml). The combined organic phases were dried over sodium sulfate and concentrated under reduced pressure. The crude title compound (1.35 g) was purified by flash chromatography [200 g of silica gel, eluant:dichloromethane/methanol=20:1 (v/v) then 8:2 (v/v)]. Evaporation of the corresponding fractions afforded 1.01 g of the pure title compound (72% yield).

Melting point: 280° C.

¹H-NMR (dmso-d₆, 200 MHz): δ=1.17 (t, 3H), 2.36 (s, 6H), 2.69 (q, 2H), 3.02, 3.04 (2 s, 6H), 4.35, 4.42 (d, bt, 3H), 4.58 (d, 2H), 6.10 (bs, 1H), 6.60 (s, 1H), 7.20 (m_{c}, 3H), 10.77 (bs, 1H).

### 7. 7-(2-Ethyl-6-methyl-benzylamino)-2,3-dimethyl-1H-pyrrolo[3,2-b]pyridine-5-carboxylic acid dimethylamide

Catalytic hydrogenation of the 3-hydroxymethyl precursor A solution of 7-(2-ethyl-6-methyl-benzylamino)-3-hydroxymethyl-2-methyl-1H-pyrrolo[3,2-b]pyridine-5-carboxylic acid dimethylamide (example 6, 200 mg, 0.52 mmol) in acetic acid (70 ml) was treated with palladium on charcoal (10 weight-%, 40 mg). A hydrogen pressure of 10 bar was applied and the reaction mixture was stirred for 3.5 hours at room temperature and for 4 hours at 50° C. The hydrogenation catalyst was removed by filtration and the filtrate was concentrated to a volume of 3 ml. Dichloromethane (50 ml) and methanol (20 ml) was added and a pH-value of 8 was adjusted with 2 N sodium hydroxide solution. The phases were separated and the aqueous phase was extracted two times with a mixture of dichloromethane (20 ml) and methanol (2 ml). The combined organic phases were evaporated to dryness and the residue (200 mg of a yellow oil) was purified by flash chromatography [70 g of silica gel, eluant:dichloromethane/methanol=20:1 (v/v)]. The pure title compound (87 mg, 46% yield) was isolated in the form of a colourless solid.

Melting point: 260-262° C.

¹H-NMR (dmso-d₆, 200 MHz): δ=1.17 (t, 3H), 2.13 (s, 3H), 2.29 (s, 3H), 2.35 (s, 3H), 2.69 (q, 2H), 3.02, 3.03 (2 s, 6H), 4.36 (d, 2H), 6.17 (bs, 1H), 6.62 (s, 1H), 7.20 (m_{c}, 3H), 10.70 (bs, 1H).

Cross-coupling of the 3-bromo precursor Four microwave reaction vessels were charged each with a suspension of 3-bromo-7-(2-ethyl-6-methyl-benzylamino)-2-methyl-1H-pyrrolo[3,2-b]pyridine-5-carboxylic acid dimethylamide (example 5, 188 mg, 0.44 mmol) in dry dioxane (4 ml). After addition of trimethylboroxine (165 mg, 183 μl, 1.31 mmol), cesium carbonate (430 mg, 1.32 mmol) and chloro-[2′-(dimethylamino)-2-biphenylyl]-(dinorbornylphosphine)-palladium (CAS 359803-53-5, 13 mg, 23 μmol), each vessel was sealed and heated to 150° C. in a microwave oven. After a period of 16 minutes, more catalyst (13 mg, 23 μmol) was added and the reaction was continued for another 16 minutes. The reaction mixtures were combined and diluted with saturated ammonium chloride solution (100 ml) and dichloromethane (100 ml). A pH-value of 7 was adjusted by addition of 3 N hydrochloric acid. The phases were separated and the aqueous phase was extracted with dichloromethane (2×50 ml). The combined organic phases were dried over sodium sulfate and evaporated to dryness. The crude product (890 mg) was purified by flash chromatography [120 g of silica gel, eluant:dichloromethane/methanol=20:1 (v/v)]. Evaporation of the corresponding fractions furnished 514 mg of a mixture of the title compound (70 weight-%, 57% yield) with 7-(2-ethyl-6-methyl-benzylamino)-2-methyl-1H-pyrrolo[3,2-b]pyridine-5-carboxylic acid dimethylamide (30 weight-%, 25% yield). The mixture was separated by preparative HPLC and the pure title compound was isolated.

### 8. (5-Chloro-2-methyl-1H-pyrrolo[3,2-b]pyridin-7-yl(2,6-dimethyl-benzyl)-amine

In a flame-dried flask filled with argon, 6-chloro-N⁴-(2,6-dimethyl-benzyl)-2-prop-1-ynyl-pyridine-3,4-diamine (example 1, 10.0 g, 33 mmol) was dissolved in dry DMF (80 ml) which had been degassed with argon. Copper(I) iodide (1.27 g, 6.7 mmol) was added and the reaction mixture was heated to 80° C. using an oil-bath which had been pre-heated to this temperature. After a reaction time of 1 hour, the hot dark-brown reaction mixture was poured onto a mixture of a 0.1 M sodium sulfite solution (160 ml) and ice (100 g). A beige suspension was obtained which was stirred for 1 hour at 0° C. The precipitate was removed by filtration and dried in vacuo (50 mbar, 60° C., 12 hours). This afforded 9.2 g of the title compound (92% yield).

Melting point: 310-312° C.

¹H-NMR (dmso-d₆, 200 MHz): δ=2.33, 2.35 (2 s, 9H), 4.32 (dd, 2H), 6.09 (bs, 1H), 6.20 (bt, 1H), 6.41 (bs, 1H), 7.16 (m_{c}, 3H), 10.77 (bs, 1H).

### 9. Ethyl[7-(2,6-dimethyl-benzylamino)-2-methyl-1H-pyrrolo[3,2-b]pyridine-5-carboxylate]

A solution of (5-chloro-2-methyl-1H-pyrrolo[3,2-b]pyridin-7-yl)-(2,6-dimethyl-benzyl)-amine (example 8, 18.9 g, 63 mmol) in ethanol (1900 ml) and DMF (475 ml) was treated with palladium(II) acetate (2.15 g, 9.6 mmol), 1,3-bis(diphenylphosphano)propane (4.47 g, 10.8 mmol), and potassium carbonate (13.2 g, 9.6 mmol). The reaction mixture was transferred into a 10 l autoclave and a carbon monoxide pressure of 18 bar was applied. The reaction mixture was heated until—after a period of 1 hour—a temperature of 200° C. and a carbon monoxide pressure of 35 bar was reached. It was kept for 3 hours at this temperature and was then cooled to room temperature over a period of 0.5 hours. The pressure was released, the reaction mixture was concentrated until most of the ethanol had been removed, and was then diluted with water (800 ml) and dichloromethane (500 ml). The phases were separated and the aqueous phase was extracted with dichloromethane (2×100 ml). The organic phases were dried over sodium sulfate and concentrated under reduced pressure. The crude product, a brown oil, was dissolved in methanol (100 ml) and silica gel (50 g) was added. The solvent was evaporated and the residue was placed on top of a column packed with 1 kg of silica gel. The title compound was eluted with mixtures of dichloromethane/methanol [30:1 (v/v), then 8:2 (v/v)]. After evaporation of the corresponding fractions two batches of the title compound were isolated: 11.42 g of beige crystals (44% corrected yield) and 5.35 g of light-brown crystals (25% yield). The first batch contained approximately 18 weight-% of DMF (as judged from the corresponding ¹H-NMR spectrum).

Melting point: 254° C.

¹H-NMR (dmso-d₆, 200 MHz): δ=1.34 (t, 3H), 2.37 (s, 9H), 4.32, 4.39 (q, d, 4H), 6.13 (bt, 1H), 6.23 (s, 1H), 7.16 (m_{c}, 4H), 10.95 (bs, 1H).

### 10. 7-(2,6-Dimethyl-benzylamino)-2-methyl-1H-pyrrolo[3,2-b]pyridine-5-carboxylic acid hydrochloride

Ethyl[7-(2,6-dimethyl-benzylamino)-2-methyl-1H-pyrrolo[3,2-b]pyridine-5-carboxylate] (example 9, 11.0 g, 33 mmol) was dissolved in methanol (210 ml) and water (21 ml). Crushed potassium hydroxide (7.3 g, 130 mmol) was added and the reaction mixture was stirred for 1.5 hours at 60° C. Most of the methanol (150 ml) was removed under reduced pressure. The reaction mixture was diluted with water (90 ml) and 6 N hydrochloric acid was added until a pH-value of 6 was obtained. A suspension was formed, which was stirred for 30 minutes at 0° C. The solid was isolated by filtration and dried in vacuo (50 mbar, 50° C., 18 h) yielding 8.05 g of the pure title compound (71% yield).

¹H-NMR (dmso-d₆, 200 MHz): δ=2.38, 2.44 (2 s, 9H), 4.62 (d, 2H), 6.36 (s, 1H), 7.20 (m_{c}, 3H), 7.31 (s, 1H), 7.96 (bt, 1H), 12.25 (s, 1H), 2 exchangeable protons not visible.

### 11. 7-(2,6-Dimethyl-benzylamino)-2-methyl-1H-pyrrolo[3,2-b]pyridine-5-carboxylic acid dimethylamide

A suspension of the hydrochloride salt of 7-(2,6-dimethyl-benzylamino)-2-methyl-1H-pyrrolo[3,2-b]pyridine-5-carboxylic acid (example 10, 11.0 g, 32 mmol) and TBTU (20.4 g, 64 mmol) in dry DMF (260 ml) was heated for 1.5 hours at 60° C. After addition of a 2 M solution of dimethylamine in THF (158 ml, 316 mmol) the reaction mixture was heated for 1 hour at 60° C. Another portion of TBTU (2.0 g, 6 mmol) and dimethylamine solution (2 M in THF, 50 ml, 100 mmol) was added. The reaction was continued for another hour at 60° C. and the solvent was removed under reduced pressure. The residue was dissolved in a mixture of dichloromethane (400 ml) and saturated sodium bicarbonate solution (400 ml). A pH-value of 10 was adjusted by addition of 6 N sodium hydroxide solution. The mixture was diluted with methanol (50 ml) and the phases were separated. The aqueous phase was extracted with a mixture of dichloromethane and methanol [1:1 (v/v), 2×100 ml]. The combined organic phases were dried over sodium sulfate and concentrated under reduced pressure. The crude product was purified by flash chromatography [150 g of silica gel, eluant:dichloromethane/methanol=20:1 (v/v)]. After evaporation of the fractions that contained the pure title compound (according to TLC analysis), 4.7 g of solid material were obtained. The solid was suspended in ethyl acetate and 2.0 g (19% yield) of the pure title compound were isolated by filtration. The chromatography fractions that contained the title compound along with impurities were combined with the filtrate and the solvent was evaporated. The residue was purified by flash chromatography [200 g of silica gel, eluant:dichloromethane/methanol=20:1 (v/v) then 10:1 (v/v)]. This afforded another 5.0 g of the pure title compound (47% yield, total yield: 66%).

Melting point: 299° C.

¹H-NMR (dmso-d₆, 200 MHz): δ=2.36 (s, 9H), 2.99, 3.00 (2 s, 6H), 4.32 (d, 2H), 5.93 (bt, 1H), 6.11 (s, 1H), 6.55 (s, 1H), 7.15 (m_{c}, 3H), 10.70 (bs, 1H).

### 12. 3-Bromo-7-(2,6-dimethyl-benzylamino)-2-methyl-1H-pyrrolo[3,2-b]pyridine-5-carboxylic acid dimethylamide

In a flask filled with argon, 7-(2,6-dimethyl-benzylamino)-2-methyl-1H-pyrrolo[3,2-b]pyridine-5-carboxylic acid dimethylamide (example 11, 2.00 g, 5.9 mmol) was dissolved in dry DMF (100 ml). The solution was cooled to 0° C., N-bromosuccinimide (1.16 g, 6.5 mmol, dissolved in 20 ml of DMF) was added over a period of 20 minutes, and the reaction was continued for 30 minutes at 0° C. The reaction solution was poured onto a mixture of dichloromethane (100 ml) and saturated sodium carbonate solution (200 ml). After addition of methanol (30 ml) the phases were separated. The aqueous phase was extracted with a mixture of dichloromethane and methanol [7:3 (v/v), 2×20 ml]. The combined organic phases were dried over sodium sulfate and concentrated in vacuo. The crude product (2.5 g) was purified by flash chromatography [100 g of silica gel, eluant:ethyl acetate/petrol ether=8:2 (v/v), then ethyl acetate/methanol=8:2 and 1:1 (v/v)] and subsequent treatment with a hot mixture of ethyl acetate (50 ml) and methanol (0.2 ml). After the suspension had been stirred for 1 hour, the title compound was removed by filtration and dried in vacuo (1.75 g, 71% yield).

Melting point: 284° C. (ethyl acetate/methanol)

¹H-NMR (dmso-d₆, 400 MHz): δ=2.35 (s, 9H), 3.02 (s, 6H), 4.34 (d, 2H), 6.07 (bt, 1H), 6.62 (s, 1H), 7.16 (m_{c}, 3H), 11.17 (bs, 1H).

### 13. 7-(2,6-Dimethyl-benzylamino)-3-hydroxymethyl-2-methyl-1H-pyrrolo[3,2-b]pyridine-5-carboxylic acid dimethylamide

In a flask filled with argon, to a suspension of 7-(2,6-dimethyl-benzylamino)-3-formyl-2-methyl-1H-pyrrolo[3,2-b]pyridine-5-carboxylic acid dimethylamide (example J, 580 mg, 1.59 mmol) in dry ethanol (12 ml), sodium borohydride (120 mg, 3.17 mmol) was added. The reaction mixture was heated to 50° C. for 5 minutes and was then stirred for 1 hour at room temperature. The solution was poured onto a mixture of dichloromethane (100 ml) and saturated ammonium chloride solution (200 ml). The biphasic mixture was stirred for 20 minutes at room temperature. The phases were separated and the aqueous phase was extracted with dichloromethane (2×20 ml). The combined organic phases were dried over sodium sulfate and concentrated under reduced pressure. The residue was suspended in a mixture of dichloromethane (10 ml) and diethyl ether (20 ml). The suspension was stirred for 30 minutes at room temperature. The pure title compound was isolated by filtration and was dried in vacuo (430 mg of a colourless solid, 74% yield).

Melting point: 301° C. (dichloromethane/diethyl ether)

¹H-NMR (dmso-d₆, 200 MHz): δ=2.36 (s, 9H), 3.02, 3.03 (2 s, 6H), 4.35 (d, 2H), 4.47 (bt, 1H), 4.57 (bd, 2H), 6.11 (bs, 1H), 6.61 (s, 1H), 7.16 (m_{c}, 3H), 10.77 (bs, 1H).

### 14. 7-(2,6-Dimethyl-benzylamino)-2,3-dimethyl-1H-pyrrolo[3,2-b]pyridine-5-carboxylic acid dimethylamide

Reduction of the 3-hydroxymethyl precursor with triethylsilane: At room temperature, triethylsilane (6.8 ml, 5.0 g, 43 mmol) was added drop-wise to a solution of 7-(2,6-dimethyl-benzylamino)-3-hydroxymethyl-2-methyl-1H-pyrrolo[3,2-b]pyridine-5-carboxylic acid dimethylamide (example 13, 1.00 g, 2.7 mmol) in trifluoroacetic acid (15 ml). The solution was stirred for 2 hours at room temperature (gas evolution was observed) and more triethylsilane (2.3 ml, 1.7 g, 14 mmol) was added. After a period of 1 hour, the reaction was poured onto a mixture of ice (100 g) and dichloromethane (50 ml). At a temperature of 0° C., a pH-value of 8.2 was adjusted by addition of 6 N sodium hydroxide solution. Addition of dichloromethane (50 ml) and methanol (20 ml) afforded a clear biphasic mixture. The phases were separated and the aqueous phase was extracted with a mixture of dichloromethane and methanol [5:1 (v/v), 2×60 ml]. The combined organic phases were dried over sodium sulfate and evaporated to dryness. The solid residue was suspended in hot ethyl acetate (50 ml). Over a period of 1 hour, the suspension was allowed to cool to room temperature. The precipitate was isolated by filtration and was washed with ethyl acetate (15 ml) and diethyl ether (15 ml). A colourless solid was obtained (630 mg), which was co-evaporated with a mixture of dichloromethane and methanol [6:1 (v/v), 35 ml] and then dried in vacuo. This afforded the pure title compound (600 mg, 63% yield).

Melting point: 319° C. (ethyl acetate)

¹H-NMR (dmso-d₆, 200 MHz): δ=2.12 (s, 3H), 2.28 (s, 3H), 2.35 (s, 6H), 3.01, 3.04 (2 s, 6H), 4.32 (d, 2H), 5.83 (bt, 1H), 6.55 (s, 1H), 7.15 (m_{c}, 3H), 10.45 (bs, 1H).

Deoxigenation of the 3-hydroxymethyl precursor via activation with O-phenyl chlorothionoformate: A suspension of 7-(2,6-dimethyl-benzylamino)-3-hydroxymethyl-2-methyl-1H-pyrrolo[3,2-b]pyridine-5-carboxylic acid dimethylamide (example 13, 200 mg, 0.55 mmol), 4-dimethylaminopyridine (20 mg, 0.16 mmol), and pyridine (90 μl, 88 mg, 1.11 mmol) in dichloromethane (5 ml) was treated with O-phenyl chlorothionoformate (129 μl, 165 mg, 0.96 mmol). A red solution was obtained which was warmed to 40° C. for 30 minutes. Equal amounts of all reagents (20 mg of 4-dimethylaminopyridine, 90 μl of pyridine, 129 μl of O-phenyl chlorothionoformate) were added and the reaction was continued for 1.5 hours at room temperature. The reaction mixture was poured onto saturated ammonium chloride solution (50 ml) and the biphasic mixture was extracted with dichloromethane (3×10 ml). The combined organic phases were dried over sodium sulfate and evaporated to dryness. This afforded crude thiocarbonic acid [7-(2,6-dimethyl-benzylamino)-5-dimethylcarbamoyl-2-methyl-1H-pyrrolo[3,2-b]pyridin-3-ylmethyl]ester phenyl ester (520 mg). After addition of dioxane (4 ml), triethylamine (0.28 ml, 0.20 g, 2.0 mmol) and hypophosphorous acid (0.22 ml, 50% in water), the reaction mixture was warmed to 100° and was treated with 2,2′-azobis(2-methylpropionitrile) [66 mg, 0.40 mmol]. After a period of 1 hour, the reaction was poured onto saturated sodium bicarbonate solution (50 ml) and was extracted with dichloromethane (3×15 ml). The combined organic phases were dried over sodium sulfate and the solvent was evaporated. The crude product was purified by flash chromatography [20 g of silica gel, eluant:dichloromethane/methanol=20:1 (v/v)]. Evaporation of the corresponding fractions afforded the pure title compound (30 mg, 15% yield).

Melting point: 315° C.

### 15. 1-Benzyl-7-(2,6-dimethyl-benzylamino)-3-hydroxymethyl-2-methyl-1H-pyrrolo[3,2-b]pyridine-5-carboxylic acid dimethylamide

Under an argon atmosphere at 0° C., 7-(2,6-dimethyl-benzylamino)-3-hydroxymethyl-2-methyl-1H-pyrrolo[3,2-b]pyridine-5-carboxylic acid dimethylamide (example 13, 100 mg, 0.27 mmol) was added to a suspension of sodium hydride (60 weight-% in oil, 11 mg, 0.28 mmol) in dry THF (3 ml). The mixture was stirred for 40 minutes at 0° C. and was evaporated to dryness. The residue was suspended in dry DMF (3 ml) and benzyl bromide (43 ml, 62 mg, 0.36 mmol) was added. The reaction mixture (a clear solution) was stirred for 1 hour at room temperature and was poured onto a mixture of saturated sodium bicarbonate solution (5 ml), water (50 ml) and dichloromethane (50 ml). The phases were separated and the aqueous phase was extracted with dichloromethane (2×10 ml). The combined organic phases were dried over sodium sulfate and concentrated in vacuo. The residue (130 mg) was purified by flash chromatography [20 g of silica gel, eluant:dichloromethane/methanol=20:1 (v/v)]. The title compound (59 mg, 48% yield) was isolated in 90% purity (as judged from the corresponding ¹H-NMR spectrum) and could be purified further by crystallization (room temperature, 20 hours) from ethyl acetate/acetic acid [20:1 (v/v), 1 ml]. The precipitate was isolated by filtration and dried in vacuo. This afforded 23 mg of the pure title compound (18% yield).

¹H-NMR (dmso-d₆, 200 MHz): δ=2.04 (s, 6H), 2.37 (s, 3H), 3.01 (s, 3H), 3.07 (s, 3H), 4.09 (d, 2H), 4.51 (t, 1H), 4.68 (m_{c}, 3H), 5.41 (s, 2H), 6.63 (m_{c}, 3H), 6.99 (m_{c}, 2H), 7.14 (m_{c}, 4H).

### 16. 7-(2,6-Dimethyl-benzylamino)-1,2,3-trimethyl-1H-pyrrolo[3,2-b]pyridine-5-carboxylic acid dimethylamide

Under an argon atmosphere at 0° C., a solution of 7-(2,6-dimethyl-benzylamino)-2,3-dimethyl-1H-pyrrolo[3,2-b]pyridine-5-carboxylic acid dimethylamide (example 14, 200 mg, 0.57 mmol) in dry DMF (7 ml) was treated with sodium hydride (60 weight-% in oil, 25 mg, 0.63 mmol). The solution was stirred for 1 hour at room temperature, cooled to 0° C., and methyl iodide (53 μl, 120 mg, 0.85 mmol) was added. The reaction mixture was stirred for 2 hours at room temperature and was poured onto a mixture of water (50 ml) and dichloromethane (50 ml). The phases were separated and the aqueous phase was extracted with dichloromethane (2×20 ml). The combined organic phases were diluted with methanol (10 ml), dried over sodium sulfate, and concentrated in vacuo. The residue (an orange oil) was purified by flash chromatography [30 g of silica gel, eluant:dichloromethane/methanol=20:1 (v/v)]. Evaporation of the corresponding fractions afforded a yellow oil (170 mg), which solidified upon treatment with diethyl ether. A suspension of the yellow solid in ethyl acetate/petrol ether [1:1 (v/v), 2 ml) was stirred for 30 minutes at room temperature. The title compound (33 mg, 16% yield) was isolated by filtration. Evaporation of the filtrate afforded 94 mg of a yellow oil, which was purified by flash chromatography [silica gel, eluant:ethyl acetate, then ethyl acetate/methanol=9:1 (v/v)] and subsequent treatment with diisopropyl ether (1 ml). This afforded another 36 mg of the pure title compound (17% yield).

Melting point: 203° C. (ethyl acetate/petrol ether)

¹H-NMR (dmso-d₆, 200 MHz): δ=2.14 (s, 3H), 2.28 (s, 3H), 2.39 (s, 6H), 3.00 (s, 3H), 3.05 (s, 3H), 3.76 (s, 3H), 4.26 (d, 2H), 5.48 (bt, 1H), 6.64 (s, 1H), 7.10 (m_{c}, 3H).

### 17. 1-Benzyl-7-(2,6-dimethyl-benzylamino)-2,3-dimethyl-1H-pyrrolo[3,2-b]pyridine-5-carboxylic acid dimethylamide

Under an argon atmosphere at 0° C., a solution of 7-(2,6-dimethyl-benzylamino)-2,3-dimethyl-1H-pyrrolo[3,2-b]pyridine-5-carboxylic acid dimethylamide (example 14, 200 mg, 0.57 mmol) in dry DMF (7 ml) was treated with sodium hydride (60 weight-% in oil, 25 mg, 0.63 mmol). The solution was stirred for 1 hour at room temperature, cooled to 0° C., and benzyl bromide (75 W, 108 mg, 0.63 mmol) was added. The reaction mixture was stirred for 1 hour at room temperature and was poured onto a mixture of saturated ammonium chloride solution (40 ml) and dichloromethane (40 ml). The phases were separated and the aqueous phase was extracted with dichloromethane (2×5 ml). The combined organic phases were dried over sodium sulfate, and concentrated in vacuo. The residue was purified by flash chromatography [20 g of silica gel, eluant:dichloromethane/methanol=40:1 (v/v)]. Evaporation of the corresponding fractions afforded a yellow oil, which solidified upon treatment with diethyl ether. A suspension of the yellow solid in ethyl acetate (0.2 ml) and diisopropyl ether (2 ml) was stirred for 20 minutes at room temperature. The title compound (95 mg, 38% yield) was isolated by filtration.

Melting point: 203° C. (ethyl acetate/diisopropyl ether)

¹H-NMR (dmso-d₆, 200 MHz): δ=2.04 (s, 6H), 2.21 (s, 3H), 2.29 (s, 3H), 3.01 (s, 3H), 3.07 (s, 3H), 4.09 (d, 2H), 4.70 (bt, 1H), 5.38 (s, 2H), 6.62 (m_{c}, 3H), 6.98 (m_{c}, 2H), 7.13 (m_{c}, 4H).

### 18. 7-(2,6-Dimethyl-benzylamino)-2-methyl-1H-pyrrolo[3,2-b]pyridine-5-carboxylic acid methylamide

A suspension of the hydrochloride salt of 7-(2,6-dimethyl-benzylamino)-2-methyl-1H-pyrrolo[3,2-b]pyridine-5-carboxylic acid (example 10, 5.0 g, 16 mmol) and TBTU (10.4 g, 32 mmol) in dry DMF (140 ml) was heated for 1 hour at 60° C. After addition of a 8 M solution of methylamine in ethanol (21 ml, 168 mmol) the reaction mixture was heated for 1 hour at 60° C. Another portion of TBTU (2.0 g, 6 mmol) and methylamine solution (8 M in ethanol, 10 ml, 80 mmol) was added. The reaction was continued for another hour at 60° C. and for 16 hours at room temperature. The solvent was removed under reduced pressure. The residue was dissolved in a mixture of dichloromethane (300 ml) and saturated sodium bicarbonate solution (300 ml). The phases were separated and the aqueous phase was extracted with dchloromethane (2×50 ml). The combined organic phases were dried over sodium sulfate and concentrated under reduced pressure. The crude product (a brown oil) was purified by flash chromatography [200 g of silica gel, eluant:dichloromethane/methanol=20:1 to 10:1 (v/v)]. Evaporation of the corresponding fractions afforded the title compound: 2.34 g of a beige solid that contained traces of DMF and tetramethyl urea (45% yield).

¹H-NMR (dmso-d₆, 200 MHz): δ=2.37 (s, 9H), 2.82 (d, 3H), 4.39 (d, 2H), 6.02 (bt, 1H), 6.18 (s, 1H), 7.16 (m_{c}, 4H), 8.51 (bq, 1H), 10.80 (bs, 1H).

### 19. Ethyl[7-(2,6-dimethyl-benzylamino)-3-hydroxymethyl-2-methyl-1H-pyrrolo[3,2-b]pyridine-5-carboxylate]

Under an argon atmosphere, a suspension of crude ethyl[7-(2,6-dimethyl-benzylamino)-3-formyl-2-methyl-1H-pyrrolo[3,2-b]pyridine-5-carboxylate] (example K, 400 mg, 1.09 mmol) in dry ethanol (8 ml) was treated at room temperature with sodium borohydride (83 mg, 2.19 mmol). The reaction mixture was stirred for 1 hour at room temperature and was poured onto a mixture of saturated ammonium chloride solution (100 ml) and dichloromethane (100 ml). The biphasic mixture was stirred for 20 minutes. The phases were separated and the aqueous phase was extracted with dichloromethane (2×5 ml). The combined organic phases were dried over sodium sulfate and the solvent was evaporated. The residue (250 mg of a yellow foam) was purified by flash chromatography [15 g of silica gel, eluant:dichloromethane/methanol=20:1 (v/v)]. The title compound was obtained in 27% yield (107 mg of a colourless solid).

Melting point: 256-259° C.

¹H-NMR (dmso-d₆, 200 MHz): δ=1.35 (t, 3H), 2.36 (s, 9H), 4.34, 4.39 (q, d, 4H), 4.50 (bt, 1H), 4.62 (d, 2H), 6.05 (bt, 1H), 7.14 (m_{c}, 4H), 10.85 (bs, 1H).

Starting Materials and Intermediates

### A. 2,6-Dichloro-4-nitroamino-pyridine

Portions of 4-amino-2,6-dichloropyridine (50.0 g, 307 mmol) were dissolved in concentrated sulphuric acid (320 ml). The rate of addition was adjusted so that an internal temperature of 10° C. was not exceeded. The mixture was cooled to −5° C. and nitric acid (90%, 150 ml) was added over a period of 40 minutes so that an internal temperature of below 0° C. was maintained. The reaction was continued for 2 hours at 0° C. and the reaction mixture was poured onto ice-water (2.5 litres, mechanical stirring). A colourless suspension was formed which was stirred for 30 minutes at 0° C. and filtered. At room temperature, the filter cake was suspended in water (1 litre) and stirring was continued for 15 minutes. The title compound was isolated by filtration and was dried in vacuo (50 mbar, 17 h, 50° C.). A colourless solid was obtained (67.0 g, quantitative yield).

¹H-NMR (dmso-d₆, 200 MHz): δ=7.48 (s, 2H), NH signal not visible.

### B. 4-Amino-2,6-dichloro-3-nitro-pyridine

Portions of 2,6-dichloro-4-nitroamino-pyridine (example A, 67.0 g, 322 mmol) were dissolved in concentrated sulphuric acid (320 ml). The rate of addition was adjusted so that an internal temperature of 40° C. was not exceeded. The reaction mixture was heated to 100° C. After a period of 1 hour, a yellow solution was obtained which was poured onto ice-water (3 litres). A pH-value of 9.5 was adjusted by addition of 6 N sodium hydroxide solution (approximately 1.9 litres). A colourless suspension was formed which was stirred for 30 minutes at room temperature. The precipitate was collected by filtration, suspended in water (4 litres), and stirring was continued for 30 minutes at room temperature. The title compound was isolated by filtration and was dried in vacuo (50 mbar, 17 h, 50 Cc). A colourless solid was obtained (75.3 g, quantitative yield).

¹H-NMR (dmso-d₆, 200 MHz): δ=6.88 (s, 1H), 7.64 (bs, 2H).

### C. (2,6-Dichloro-3-nitro-pyridin-4-yl)-(2-ethyl-6-methyl-benzyl)-amine

In a flame-dried flask filled with nitrogen, sodium hydride (5.2 g, 60 weight-% in paraffin, 130 mmol) was suspended in dry THF (120 ml). A solution of 4-amino-2,6-dichloro-3-nitropyridine (example B, 22.4 g, 108 mmol) in dry THF (200 ml) was added drop-wise so that a temperature of 10-15 Cc was maintained in the flask. A red solution was obtained which was stirred for 1 hour. A second flask was filled with nitrogen, charged with a solution of 2-ethyl-6-methylbenzyl chloride (20.0 g, 118 mmol) in dry THF (120 ml), and sodium iodide (17.7 g, 118 mmol) was added. A yellow suspension was obtained which was stirred for 1 hour and was then slowly added to the content of the first flask so that a temperature of 10° C. was not exceeded. The reaction mixture was stirred for 1 hour at 0° C., poured onto ice-water (1200 ml), and extracted with ethyl acetate (2×400 ml, 2×200 ml). The organic phases were dried over sodium sulfate and concentrated to a volume of 600 ml. Silica gel (100 g) was added and the remaining solvent was removed in vacuo. A flash column prepared with 1.2 kg of silica gel was charged with the residue and the title compound was eluted with petrol ether/ethyl acetate 15:1 (v/v). A colourless solid (26.3 g, 77% yield) was obtained which was pure by means of ¹H-NMR spectroscopy.

Melting point: 128-129° C. (petrol ether/ethyl acetate)

¹H-NMR (CDCl₃, 200 MHz): δ=1.22 (t, 3H), 2.35 (s, 3H), 2.67 (q, 2H), 4.36 (d, 2H), 6.23 (bt, 1H), 6.86 (s, 1H), 7.19 (m_{c}, 3H).

### D. (6-Chloro-3-nitro-2-prop-1-ynyl-pyridin-4-yl)-(2-ethyl-6-methyl-benzyl)-amine

In a flame-dried flask filled with argon, a solution of (2,6-dichloro-3-nitro-pyridin-4-yl)-(2-ethyl-6-methylbenzyl)-amine (example C, 10.0 g, 29 mmol) in dry 1,4-dioxane (30 ml) was treated with tri-n-butyl-1-propynylstannane (9.9 ml, 10.7 g, 33 mmol) and bis(triphenylphosphine)palladium(II) dichloride (1.00 g, 1.4 mmol). The reaction mixture was heated to 50° C., kept at this temperature for 3 hours, and evaporated in the presence of silica gel (60 g). A flash column filled with 500 g of silica gel was charged with the residue. After the organotin compounds had been removed by elution with petrol ether (1 l), the title compound was eluted with petrol ether/ethyl acetate=10:1 (v/v). Orange crystals (7.7 g, 76% yield) were isolated. As indicated by the ¹H-NMR spectrum, organotin compounds were removed almost completely in the course of the work-up/purification.

Melting point: 133° C.

¹H-NMR (CDCl₃, 200 MHz): δ=1.22 (t, 3H), 2.13 (s, 3H), 2.35 (s, 3H), 2.67 (q, 2H), 4.35 (d, 2H), 6.82 (s, 1H), 6.92 (bt, 1H), 7.19 (m_{c}, 3H).

### E. 6-Chloro-N⁴-(2-ethyl-6-methyl-benzyl)-2-prop-1-ynyl-pyridine-3,4-amine

A solution of (6-chloro-3-nitro-2-prop-1-ynyl-pyridin-4-yl)-(2-ethyl-6-methyl-benzyl)-amine (example D, 10.0 g, 29 mmol) in diethyl ether (140 ml) was treated with a solution of tin(II) chloride (66.0 g, 293 mmol) in concentrated hydrochloric acid (34.4 ml). The rate of the addition was adjusted so that gentle boiling of the etherous solution was maintained; approximately 15 minutes were required for complete addition of the reagent. The reaction mixture was stirred for 1 hour at room temperature, poured onto a mixture of ice-water (1 l) and ethyl acetate (500 ml), and the pH-value of the biphasic mixture was adjusted to 10 by addition of 6 N sodium hydroxide solution. The suspension was filtered over Celite 545, the filter cake was washed with ethyl acetate (400 ml), and the phases of the obtained filtrate were separated. The aqueous phase was extracted with ethyl acetate (2×200 ml). The combined organic phases were dried over sodium sulfate and evaporated to dryness yielding 5.38 g of title compound (beige solid, 59% yield). Further 3.4 g of the title compound (colourless solid, 37% yield) were obtained by continuous extraction of the Celite residue with ethyl acetate (1 l) using a Soxhlet apparatus (15 h) and subsequent concentration in vacuo.

Melting point: 218° C. (petrol ether/ethyl acetate)

¹H-NMR (CDCl₃, 200 MHz): δ=1.23 (t, 3H), 2.11 (s, 3H), 2.37 (s, 3H), 2.69 (q, 2H), 3.48 (bs, 2H), 3.88 (bt, 1H), 4.24 (d, 2H), 6.59 (s, 1H), 7.18 (m_{c}, 3H).

### F. 7-(2-Ethyl-6-methyl-benzylamino)-3-formyl-2-methyl-1H-pyrrolo[3,2-b]pyridine-5-carboxylic acid dimethylamide

A flask filled with argon was charged with dry DMF (7 ml) and phosphorous oxychloride (660 μl, 1.10 g, 7.2 mmol) was added at a temperature of 0° C. The solution was stirred for 1 hour at room temperature and was then added at 0° C. to a solution of 7-(2-ethyl-6-methyl-benzylamino)-2-methyl-1H-pyrrolo[3,2-b]pyridine-5-carboxylic acid dimethylamide (example 4, 1.00 g, 2.9 mmol) in dry DMF (10 ml). The reaction mixture was stirred for 20 hours at room temperature and more Vilsmeier reagent (prepared from 660 μl of phosphorous oxychloride and 7 ml of dry DMF as described above) was added at 0° C. The reaction was continued for 16 hours at room temperature and was quenched by addition of ice-water (50 ml). Dichloromethane (100 ml) and saturated sodium bicarbonate solution (50 ml) was added and a pH-value of 8 was adjusted with 2 N sodium hydroxide solution. The phases were separated and the aqueous phase was extracted with dichloromethane (2×30 ml). The combined organic phases were dried over sodium sulfate and concentrated under reduced pressure. The residue (1.2 g of a yellow oil) was purified by flash chromatography [60 g of silica gel, eluant:ethyl acetate/methanol=10:1 (v/v)]. Evaporation of the corresponding fractions afforded the pure title compound (620 mg of yellow crystals, 58% yield).

¹H-NMR (dmso-d₆, 200 MHz): δ=1.17 (t, 3H), 2.36 (s, 3H), 2.63, 2.70 (s, q, 5H), 3.02 (s, 6H), 4.35 (d, 2H), 6.25 (bt, 1H), 6.70 (s, 1H), 7.20 (m_{c}, 3H), 10.26 (s, 1H), 11.91 (bs, 1H).

### G. (2,6-Dichloro-3-nitro-pyridin-4-yl)-(2,6-dimethyl-benzyl)-amine

In a flame-dried flask filled with nitrogen, sodium hydride (4.8 g, 60 weight-% in paraffin, 120 mmol) was suspended in dry THF (110 ml). The grey suspension was cooled to 0° C. and a solution of 4-amino-2,6-dichloro-3-nitropyridine (example B, 25.0 g of crude product, 120 mmol) in dry THF (180 ml) was added drop-wise so that a temperature of 10-15° C. was maintained in the flask. A red solution was obtained which was stirred for 1 hour at 0° C. A second flask was filled with nitrogen, charged with a solution of 2,6-dimethylbenzyl chloride (17.0 g, 110 mmol) in dry THF (110 ml), and sodium iodide (16.5 g, 110 mmol) was added. A colourless suspension was obtained which was stirred for 1 hour at room temperature and was then slowly added to the content of the first flask so that a temperature of 5° C. was not exceeded. The reaction mixture was stirred for 1 hour at 0° C., poured onto ice-water (1500 ml), and extracted with ethyl acetate (3×250 ml). The organic phases were dried over sodium sulfate and concentrated to a volume of 400 ml. Silica gel (100 g) was added and the remaining solvent was removed in vacuo. A flash column prepared with 1 kg of silica gel was charged with the residue and the title compound was eluted with petrol ether/ethyl acetate [15:1 (v/v), then 10:1 (v/v)]. The title compound was obtained as a yellow solid (30.8 g, 79% yield), pure by means of ¹H-NMR spectroscopy.

Melting point: 173-174° C.

¹H-NMR (dmso-d₆, 200 MHz): δ=2.29 (s, 6H), 4.38 (d, 2H), 7.08 (m_{c}, 3H), 7.20 (s, 1H), 7.72 (bt, 1H).

### H. (6-Chloro-3-nitro-2-prop-1-ynyl-pyridin-4-yl)-(2,6-dimethyl-benzyl)-amine

In a flame-dried flask filled with argon, (2,6-dichloro-3-nitro-pyridin-4-yl)-(2,6-dimethyl-benzyl)-amine (example G, 38.0 g, 117 mmol) and bis(triphenylphosphine)palladium(II) dichloride (4.10 g, 5.8 mmol) were dissolved in dry 1,4-dioxane (120 ml) and tri-n-butyl-1-propynylstannane (37.2 ml, 40.3 g, 122 mmol) was added. The reaction mixture was heated to 50° and was kept at this temperature for 3 hours. More bis(triphenylphosphine)palladium(II) dichloride (0.50 g, 0.7 mmol) and tri-n-butyl-1-propynylstannane (0.9 ml, 1.0 g, 3 mmol) was added and the reaction was continued for 1.5 hours at 50° C. The brown solution was evaporated in the presence of silica gel (80 g). A flash column filled with 1000 g of silica gel was charged with the residue. After the organotin compounds had been removed by elution with petrol ether (2 litres), the title compound was eluted with petrol ether/ethyl acetate=10:1 (v/v) and ethyl acetate. Evaporation of the corresponding fractions furnished two batches of the title compound: a beige solid (22.3 g, 58% yield) and a brown oil (19 g). The second batch was further purified by column chromatography [500 g of silica gel, eluant:petrol ether/ethyl acetate=5:1 (v/v)]. A beige solid (9.9 g, 26% yield) was isolated. The amount of organotin compounds present in the two batches (13 mol-%/4 mol %) was determined by ¹H-NMR spectroscopy.

Melting point (second batch): 146-148° C.

¹H-NMR (dmso-d₆, 400 MHz): δ=2.08 (s, 3H), 2.30 (s, 6H), 4.37 (d, 2H), 7.11 (m_{c}, 4H), 7.37 (bt, 1H), signals of tri-n-butylstannane at 6=0.88 (t), 1.15 (m_{c}), 1.31 (m_{c}), 1.61 (m_{c}).

### I. 6-Chloro-N⁴-(2,6-dimethyl-benzyl)-2-prop-1-ynyl-pyridine-3,4-diamine

A solution of (6-chloro-3-nitro-2-prop-1-ynyl-pyridin-4-yl)-(2,6-dimethyl-benzyl)-amine (example H, 10.0 g, 30 mmol) in diethyl ether (140 ml) was treated with a solution of tin(II) chloride dihydrate (69.0 g, 306 mmol) in concentrated hydrochloric acid (36.0 ml). The rate of the addition was adjusted so that gentle boiling of the etherous solution was maintained; approximately 15 minutes were required for complete addition of the reagent. The reaction mixture was stirred for 1 hour at room temperature, poured onto a mixture of ice-water (1 litre) and ethyl acetate (500 ml), and the pH-value of the biphasic mixture was adjusted to 10 by addition of 6 N sodium hydroxide solution. The suspension was filtered over Celite 545, the filter cake was washed with ethyl acetate (2×150 ml), and the phases of the obtained filtrate were separated. The aqueous phase was extracted with ethyl acetate (2×150 ml). The combined organic phases were dried over sodium sulfate and evaporated to dryness. The brown solid residue (3.78 g) was treated with hot diethyl ether (15 ml). The suspension was allowed to cool to room temperature, diluted with more diethyl ether (40 ml), and stirring was continued for 1 hour at 0° C. The title compound was isolated by filtration (2.0 g of yellow crystals, 22% yield). The Celite residue was continuously extracted with a mixture of ethyl acetate/methanol=9:1 (v/v) [1 litre] using a Soxhlet apparatus. After a period of 15 hours, the solvent was evaporated. The yellow solid residue (7.0 g) was suspended in boiling chloroform (300 ml). The hot suspension was stirred for 1 hour at reflux and was filtered rapidly. Concentration of the filtrate afforded a second batch of the title compound (5.14 g of a yellow solid, 56% yield). Both batches contained only traces of organotin impurities (as confirmed by ¹H-NMR spectroscopy).

Melting point: 272-274° C. (chloroform)

¹H-NMR (dmso-d₆, 200 MHz): δ=2.09 (s, 3H), 2.31 (2 s, 6H), 4.19 (d, 2H), 5.09 (bs, 2H), 5.74 (bt, 1H), 6.50 (s, 1H), 7.12 (m_{c}, 3H).

### J. 7-(2,6-Dimethyl-benzylamino)-3-formyl-2-methyl-1H-pyrrolo[3,2-b]pyridine-5-dimethylamide

In a flask filled with argon, phosphorous oxychloride (0.72 ml, 1.21 g, 7.9 mmol) was added to a suspension of 7-(2,6-dimethyl-benzylamino)-2-methyl-1H-pyrrolo[3,2-b]pyridine-5-carboxylic acid dimethylamide (example 11, 1.30 g, 3.9 mmol) in dry DMF (8 ml). The reaction mixture was heated to 80° C. for 1 hour. After addition of another portion of phosphorous oxychloride (0.10 ml, 0.17 g, 1.1 mmol) the reaction was continued for 40 minutes at 80° C. The brown solution was added slowly to an ice-cold mixture of sodium bicarbonate solution (600 ml) and dichloromethane (200 ml). The biphasic mixture was stirred for 20 minutes. The phases were separated and the aqueous phase was extracted with dichloromethane (2×50 ml). The combined organic phases were dried over sodium sulfate and the solvent was evaporated. To a solution of the crude product in DMF (3 ml), dichloromethane (5 ml), and diethyl ether (10 ml) was added. The resulting suspension was stirred for 30 minutes at room temperature and the precipitate was isolated by filtration. This afforded the pure title compound (600 mg, 43% yield).

Note: It is also possible to omit the crystallization step and to subject the crude product (which contains about 60 weight-% of the title compound) to the reduction step described in example 13.

¹H-NMR (dmso-d₆, 200 MHz): δ=2.36 (s, 6H), 2.63 (s, 3H), 3.02 (s, 6H), 4.35 (d, 2H), 6.15 (bt, 1H), 6.70 (s, 1H), 7.17 (m_{c}, 3H), 10.26 (s, 1H), 11.72 (bs, 1H).

### K. Ethyl[7-(2,6-dimethyl-benzylamino)-3-formyl-2-methyl-1H-pyrrolo[3,2-b]pyridine-5-carboxylate]

In a flask filled with argon, phosphorous oxychloride (0.28 ml, 0.47 g, 3.1 mmol) was added to a suspension of ethyl[7-(2,6-dimethyl-benzylamino)-2-methyl-1H-pyrrolo[3,2-b]pyridine-5-carboxylate] (example 9, 0.50 g, 1.5 mmol) in dry DMF (3 ml). A dark brown solution was obtained, which was stirred for 20 minutes at room temperature and for 40 minutes at 80° C. The reaction solution was poured slowly onto 50 ml of ice-water. Dichloromethane (50 ml) was added and a pH-value of 6 was adjusted with 2 N sodium hydroxide solution. The phases were separated and the aqueous phase was extracted with dichloromethane (2×10 ml). The combined organic phases were dried over sodium sulfate and the solvent was evaporated. The brown oily residue was purified by flash chromatography [30 g of silica gel, eluant:dichloromethane/methanol=20:1 to 6:4 (v/v)] The title compound (400 mg, 74% yield) was isolated in 80% purity (as judged from the corresponding ¹H-NMR spectrum).

¹H-NMR (dmso-d₆, 200 MHz): δ=1.37 (t, 3H), 2.38 (s, 6H), 2.66 (s, 3H), 4.37 (m_{c}, 4H), 6.74 (bt, 1H), 7.14 (m_{c}, 3H), 7.29 (s, 1H), 10.25 (s, 1H), 12.53 (bs, 1H).

### L. 6-Chloro-3-nitro-2-prop-1-ynyl-pyridin-4-ylamine

In a flame-dried flask filled with argon, a solution of 4-amino-2,6-dichloro-3-nitropyridine (example B, 15.0 g, 72 mmol) in dry 1,4-dioxane (60 ml) was treated with tri-n-butyl-1-propynylstannane (23.1 ml, 25.0 g, 76 mmol) and bis(triphenylphosphine)palladium(II) dichloride (2.53 g, 3.6 mmol). The reaction mixture was heated to 50° C., kept at this temperature for 4 hours, and evaporated in the presence of silica gel (50 g). A flash column filled with 400 g of silica gel was charged with the residue. After most of the organotin compounds had been removed by elution with petrol ether (3 l), the title compound was eluted with petrol ether/ethyl acetate=7:3 and 1:1 (v/v). Evaporation of the corresponding fractions afforded a brown solid, which was dried in vacuo (16.1 g) and analysed by ¹H-NMR spectroscopy as a mixture of the title compound (63 mol-%), untransformed starting material (27 mol-%), organotin compounds (7 mol-%) and ethyl acetate (3 mol-%). The title compound was used without further purification for the reduction step described in example M.

¹H-NMR (dmso-d₆, 200 MHz, mixture, only signals of the title compound reported): δ=2.09 (s, 3H), 6.84 (s, 1H), 7.50 (bs, 2H).

### M. 6-Chloro-2-prop-1-ynyl-pyridine-3,4-diamine

A solution of 6-chloro-3-nitro-2-prop-1-ynyl-pyridin-4-ylamine (example L, 16.0 g of crude product, 48 mmol) in diethyl ether (120 ml) was treated with a solution of tin(II) chloride dihydrate (160.0 g, 709 mmol) in concentrated hydrochloric acid (60 ml). The rate of the addition was adjusted so that gentle boiling of the etherous solution was maintained; approximately 15 minutes were required for complete addition of the reagent. The reaction mixture was stirred for 1 hour at room temperature, poured onto a mixture of ice-water (1 litre) and ethyl acetate (600 ml), and the pH-value of the biphasic mixture was adjusted to 9.6 by addition of 6 N sodium hydroxide solution. The dense suspension was stirred for 2 hours at room temperature and was filtered over Celite 545. The phases of the obtained filtrate were separated and the aqueous phase was extracted with ethyl acetate (2×200 ml). The combined organic phases were dried over sodium sulfate and evaporated to dryness. The yellow solid residue (10.5 g) was dissolved in a mixture of dichloromethane and methanol and the solvent was evaporated in the presence of 30 g of silica gel. A flash column filled with 400 g of silica gel was charged with the residue. The pure title compound was isolated by elution with ethyl acetate/petrol ether=1:1 (v/v) and evaporation of the corresponding fractions (6.0 g of yellow crystals, 69% yield).

Melting point: 145-146° C.

¹H-NMR (dmso-d₆, 200 MHz): δ=2.09 (s, 3H), 4.85 (bs, 2H), 5.91 (bs, 2H), 6.37 (s, 1H).

### N. 5-Chloro-2-methyl-1H-pyrrolo[3,2-b]pyridin-7-ylamine

In a flame-dried flask filled with argon, 6-chloro-2-prop-1-ynyl-pyridine-3,4-diamine (example M, 2.0 g, 11 mmol) was dissolved in dry DMF (20 ml). Copper(I) iodide (420 mg, 2.20 mmol) was added and the reaction mixture was heated to 80° C. using an oil-bath which had been pre-heated to this temperature. After a reaction time of 1 hour, the solvent was evaporated under reduced pressure. The residue was dissolved in water (100 ml) and dichloromethane (70 ml). The phases were separated and the aqueous phase was extracted with dichloromethane (2×70 ml). The combined organic phases were diluted with methanol (30 ml), dried over sodium sulfate, and evaporated to dryness. A dark-brown oily residue (2.4 g) was isolated, which was purified by flash chromatography [100 g of silica gel, eluant:dichloromethane/methanol=20:1 to 7:3 (v/v)]. The pure title compound was obtained after evaporation of the corresponding fractions (1.35 g of yellow crystals, 68% yield).

Melting point: 270° C.

¹H-NMR (dmso-d₆, 200 MHz): δ=2.38 (s, 3H), 6.04 (bs, 3H), 6.23 (bs, 1H), 10.70 (bs, 1H).

Commercial Utility

The compounds of the formula 1 and 1-1 and their salts (active compounds according to the invention) have valuable pharmacological properties which make them commercially utilizable. In particular, they exhibit marked inhibition of gastric acid secretion and an excellent gastric and intestinal protective action in warm-blooded animals, in particular humans. In this connection, the active compounds according to the invention are distinguished by a high selectivity of action, an advantageous duration of action, a particularly good enteral activity, the absence of significant side effects and a large therapeutic range.

“Gastric and intestinal protection” in this connection is understood as meaning the prevention and treatment of gastrointestinal diseases, in particular of gastrointestinal inflammatory diseases and lesions (such as, for example, gastric ulcer, peptic ulcer, including peptic ulcer bleeding, duodenal ulcer, gastritis, hyperacidic or medicament-related functional dyspepsia), which can be caused, for example, by microorganisms (e.g. Helicobacter pylori), bacterial toxins, medicaments (e.g. certain antiinflammatories and antirheumatics, such as NSAIDs and COX-inhibitors), chemicals (e.g. ethanol), gastric acid or stress situations. “Gastric and intestinal protection” is understood to include, according to general knowledge, gastroesophageal reflux disease (GERD), the symptoms of which include, but are not limited to, heartburn and/or acid regurgitation.

In their excellent properties, the active compounds according to the invention surprisingly prove to be clearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the abovementioned diseases.

The invention furthermore includes the use of the active compounds according to the invention for the treatment and/or prophylaxis of the abovementioned diseases.

A further subject of the invention are medicaments which comprise one or more compounds of the active compounds according to the invention.

The medicaments are prepared by processes which are known per se and familiar to the person skilled in the art. As medicaments, the pharmacologically active compounds according to the invention are either employed as such, or preferably in combination with suitable pharmaceutical auxiliaries or excipients in the form of tablets, coated tablets, capsules, suppositories, patches (e.g. as TTS), emulsions, suspensions or solutions, the active compound content advantageously being between 0.1 and 95% and it being possible to obtain a pharmaceutical administration form exactly adapted to the active compound and/or to the desired onset and/or duration of action (e.g. a sustained-release form or an enteric form) by means of the appropriate selection of the auxiliaries and excipients.

The auxiliaries and excipients which are suitable for the desired pharmaceutical formulations are known to the person skilled in the art on the basis of his/her expert knowledge. In addition to solvents, gel-forming agents, suppository bases, tablet auxiliaries and other active compound excipients, it is possible to use, for example, antioxidants, dispersants, emulsifiers, antifoams, flavor corrigents, preservatives, solubilizers, colorants or, in particular, permeation promoters and complexing agents (e.g. cyclodextrins).

The active compounds according to the invention can be administered orally, parenterally or percutaneously.

In general, it has proven advantageous in human medicine to administer the active compound according to the invention in the case of oral administration in a daily dose of approximately 0.01 to approximately 20, preferably 0.05 to 5, in particular 0.1 to 1.5, mg/kg of body weight, if appropriate in the form of several, preferably 1 to 4, individual doses to achieve the desired result. In the case of a parenteral treatment, similar or (in particular in the case of the intravenous administration of the active compounds), as a rule, lower doses can be used. The establishment of the optimal dose and manner of administration of the active compounds necessary in each case can easily be carried out by any person skilled in the art on the basis of his/her expert knowledge.

If the active compound according to the invention and/or their salts are to be used for the treatment of the abovementioned diseases, the pharmaceutical preparations can also contain one or more pharmacologically active constituents of other groups of medicaments, for example: tranquillizers (for example from the group of the benzodiazepines, for example diazepam), spasmolytics (for example, bietamiverine or camylofine), anticholinergics (for example, oxyphencyclimine or phencarbamide), local anesthetics, (for example, tetracaine or procaine), and, if appropriate, also enzymes, vitamins or amino acids.

To be emphasized in this connection is in particular the combination of the active compounds according to the invention with pharmaceuticals which inhibit acid secretion, such as, for example, H₂ blockers (e.g. cimetidine, ranitidine), H⁺/K⁺ ATPase inhibitors (e.g. omeprazole, pantoprazole), or further with so-called peripheral anticholinergics (e.g. pirenzepine, telenzepine) and with gastrin antagonists with the aim of increasing the principal action in an additive or super-additive sense and/or of eliminating or of decreasing the side effects, or further the combination with antibacterially active substances (such as, for example, cephalosporins, tetracyclines, penicillins, macrolides, nitroimidazoles or alternatively bismuth salts) for the control of Helicobacter pylori. Suitable antibacterial co-components which may be mentioned are, for example, mezlocillin, ampicillin, amoxicillin, cefalothin, cefoxitin, cefotaxime, imipenem, gentamycin, amikacin, erythromycin, ciprofloxacin, metronidazole, clarithromycin, azithromycin and combinations thereof (for example clarithromycin+metronidazole).

In view of their excellent gastric and intestinal protection action, the active compounds according to the invention are suited for a free or fixed combination with those medicaments (e.g. certain antiinflammatories and antirheumatics, such as NSAIDs), which are known to have a certain ulcerogenic potency. In addition, the active compounds according to the invention are suited for a free or fixed combination with motility-modifying drugs.

Pharmacology

The excellent gastric protective action and the gastric acid secretion-inhibiting action of the compounds of the formula 1 according to the invention can be demonstrated in investigations on animal experimental models. The compounds according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal/intravenous administration in vivo is shown.

TABLE A

| | Dose | Dose | |
|---|---|---|---|
| | (μmol/kg) | (μmol/kg) | Inhibition of acid secretion |
| No. | i.d. | i.v. | (%) |
| 5 | 1 | | >30% |
| 6 | 1 | | >30% |
| 7 | | 2 | >80% |
| 12 | 3 | | >60% |
| 14 | 1 | | >50% |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was continuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 ml/h) of i.v. pentagastrin (left femoral vein) about 30 min after the end of the operation (i.e. after determination of 2 preliminary fractions). The substances to be tested were administered intraduodenally (substance 7 intravenously) in a 2.5 ml/kg liquid volume 60 min after the start of the continuous pentagastrin infusion.

The body temperature of the animals was kept at a constant 37.8-38° C. by infrared irradiation and heat pads (automatic, stepless control by means of a rectal temperature sensor).

1. A compound of the formula 1 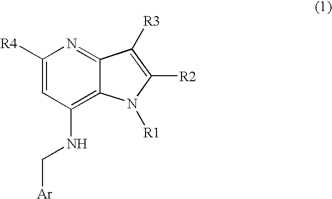
in which
R1 is hydrogen, a radical CH₂—R11 or a radical CH(OR12)-R11,
where
R11 is hydrogen, 1-4C-alkyl or a group Cy,
R12 is hydrogen, 1-4C-alkyl, 3-7C-cycloalkyl, 3-7C-cycloalkyl-1-4C-alkyl, 1-4C-alkoxy-1-4C-alkyl, 1-4C -alkoxycarbonyl, fluoro-1-4C-alkyl, 1-4C-alkylcarbonyl or the radical —CO—N(R121)(R122),
where
R121 is hydrogen, 1-7C-alkyl, hydroxy-1-4C-alkyl, 1-4C-alkoxy-1-4C-alkyl or 3-7C-cycloalkyl and
R122 is hydrogen or 1-7C-alkyl,
or where
R121 and R122 together and including the nitrogen atom to which they are attached form a pyrrolidino, piperidino, morpholino, aziridino or azetidino radical,
R2 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl, and
R32 is hydrogen or 1-7C-alkyl,
or where
R31 and R32 together and including the nitrogen atom to which they are attached form a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
where
R41 is hydrogen, 1-7C-alkyl, hydroxy-1-4C-alkyl, 1-4C-alkoxy-1-4C-alkyl or 3-7C-cycloalkyl, and
R42 is hydrogen or 1-7C-alkyl,
or where
R41 and R42 together and including the nitrogen atom to which they are attached are a pyrrolidino, piperidino, morpholino, aziridino or azetidino radical, optionally substituted by a hydroxy radical,
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxyl,
R7 is hydrogen, 1-4C-alkyl, 1-4C-alkoxy, hydroxyl or halogen and
R8 is hydrogen, 1-4C-alkyl or halogen,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
and Cy is a tetrahydrofuryl group or a mono- or bicyclic aromatic residue as defined for Ar or aryl,
or a salt thereof.

2. A compound of the formula 1 as claimed in claim 1, in which
R1 is hydrogen or a radical CH₂—R11,
where
R11 is hydrogen, 1-4C-alkyl, aryl, tetrahydrofuryl, pyridyl, furyl or thienyl,
R2 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl, and
R32 is hydrogen or 1-7C-alkyl,
or where
R31 and R32 together and including the nitrogen atom to which they are attached form a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
where
R41 is hydrogen, 1-7C-alkyl, hydroxy-1-4C-alkyl, 1-4C-alkoxy-1-4C-alkyl or 3-7C-cycloalkyl and
R42 is hydrogen or 1-7C-alkyl,
or where
R41 and R42 together and including the nitrogen atom to which they are attached are a pyrrolidino, piperidino, morpholino, aziridino or azetidino radical,
Ar is a mono- or bicyclic aromatic residue, substituted by R5, R6, R7 and R8, which is selected from the group consisting of phenyl, naphthyl, pyrrolyl, pyrazolyl, imidazolyl, 1,2,3-triazolyl, indolyl, benzimidazolyl, furyl, benzofuryl, thienyl, benzothienyl, thiazolyl, isoxazolyl, pyridinyl, pyrimidinyl, chinolinyl and isochinolinyl,
wherein
R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxyl,
R7 is hydrogen, 1-4C-alkyl, 1-4C-alkoxy, hydroxyl or halogen and
R8 is hydrogen, 1-4C-alkyl or halogen,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
or a salt thereof.

3. A compound of the formula 1 as claimed in claim 1, in which
R1 is hydrogen, a radical CH₂—R11, or a radical CH(OR12)-R11,
where
R11 is hydrogen, 1-4C-alkyl, aryl, tetrahydrofuryl, pyridyl, furyl or thienyl,
R12 is hydrogen, 1-4C-alkyl, 3-7C-cycloalkyl, 3-7C-cycloalkyl-1-4C-alkyl, 1-4C-alkoxy-1-4C-alkyl or fluoro-1-4C-alkyl,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is hydrogen or 1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
where
R31 is hydrogen, 1-7C-alkyl, hydroxy-1-4C-alkyl, 1-4C-alkoxy-1-4C-alkyl, or 3-7C-cycloalkyl, and
R32 is hydrogen or 1-7C-alkyl,
or where
R31 and R32 together and including the nitrogen atom to which they are attached form a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
where
R41 is hydrogen, 1-7C-alkyl, hydroxy-1-4C-alkyl, 1-4C-alkoxy-1-4C-alkyl, or 3-7C-cycloalkyl and
R42 is hydrogen or 1-7C-alkyl,
or where
R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6,
where
R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
or
Ar is selected from the group consisting of 4-acetoxyphenyl, 4-acetamidophenyl, 2-methoxyphenyl, 3-methoxyphenyl, 4-methoxyphenyl, 3-benzyloxyphenyl, 4-benzyloxyphenyl, 3-benzyloxy-4-methoxyphenyl, 4-benzyloxy-3-methoxyphenyl, 3,5-bis-(trifluoromethyl)phenyl, 4-butoxyphenyl, 2-chlorophenyl, 3-chlorophenyl, 4-chlorophenyl, 2-chloro-6-fluorophenyl, 3-chloro-4-fluorophenyl, 2-chloro-5-nitrophenyl, 4-chloro-3-nitrophenyl, 3-(4-chlorophenoxy)phenyl, 2,4-dichlorophenyl, 3,4-difluorophenyl, 2,4-dihydroxyphenyl, 2,6-dimethoxyphenyl, 3,4-dimethoxy-5-hydroxyphenyl, 2,5-dimethylphenyl, 3-ethoxy-4-hydroxyphenyl, 2-fluorophenyl, 4-fluorophenyl, 4-hydroxyphenyl, 2-hydroxy-5-nitrophenyl, 3-methoxy-2-nitrophenyl, 3-nitrophenyl, 2,3,5-trichlorophenyl, 2,4,6-trihydroxyphenyl, 2,3,4-trimethoxyphenyl, 2-hydroxy-1-naphthyl, 2-methoxy-1-naphthyl, 4-methoxy-1-naphthyl, 1-methyl-2-pyrrolyl, 2-pyrrolyl, 3-methyl-2-pyrrolyl, 3,4-dimethyl-2-pyrrolyl, 4-(2-methoxycarbonylethyl)-3-methyl-2-pyrrolyl, 5-ethoxycarbonyl-2,4-dimethyl-3-pyrrolyl, 3,4-dibromo-5-methyl-2-pyrrolyl, 2,5-dimethyl-1-phenyl-3-pyrrolyl, 5-carboxy-3-ethyl-4-methyl-2-pyrrolyl, 3,5-dimethyl-2-pyrrolyl, 2,5-dimethyl-1-(4-trifluoromethylphenyl)-3-pyrrolyl, 1-(2,6-dichloro-4-trifluoromethylphenyl)-2-pyrrolyl, 1-(2-nitrobenzyl)-2-pyrrolyl, 1-(2-fluorophenyl)-2-pyrrolyl, 1-(4-trifluoromethoxyphenyl)-2-pyrrolyl, 1-(2-nitrobenzyl)-2-pyrrolyl, 1-(4-ethoxycarbonyl)-2,5-dimethyl-3-pyrrolyl, 5-chloro-1,3-dimethyl-4-pyrazolyl, 5-chloro-1-methyl-3-trifluoromethyl-4-pyrazolyl, 1-(4-chlorobenzyl)-5-pyrazolyl, 1,3-dimethyl-5-(4-chlorophenoxy)-4-pyrazolyl, 1-methyl-3-trifluoromethyl-5-(3-trifluoromethylphenoxy)-4-pyrazolyl, 4-methoxycarbonyl-1-(2,6-dichlorophenyl)-5-pyrazolyl, 5-allyloxy-1-methyl-3-trifluoromethyl-4-pyrazolyl, 5-chloro-1-phenyl-3-trifluoromethyl-4-pyrazolyl, 3,5-dimethyl-1-phenyl-4-imidazolyl, 4-bromo-1-methyl-5-imidazolyl, 2-butylimidazolyl, 1-phenyl-1, 2,3-triazol-4-yl, 3-indolyl, 4-indolyl, 7-indolyl, 5-methoxy-3-indolyl, 5-benzyloxy-3-indolyl, 1-benzyl-3-indolyl, 2-(4-chlorophenyl)-3-indolyl, 7-benzyloxy-3-indolyl, 6-benzyloxy-3-indolyl, 2-methyl-5-nitro-3-indolyl, 4,5,6,7-tetrafluoro-3-indolyl, 1-(3,5-difluorobenzyl)-3-indolyl, 1-methyl-2-(4-trifluorophenoxy)-3-indolyl, 1-methyl-2-benzimidazolyl, 5-nitro-2-furyl, 5-hydroxymethyl-2-furyl, 2-furyl, 3-furyl, 5-(2-nitro-4-trifluoromethylphenyl)-2-furyl, 4-ethoxycarbonyl-5-methyl-2-furyl, 5-(2-trifluoromethoxyphenyl)-2-furyl, 5-(4-methoxy-2-nitrophenyl)-2-furyl, 4-bromo-2-furyl, 5-dimethylamino-2-furyl, 5-bromo-2-furyl, 5-sulfo-2-furyl, 2-benzofuryl, 2-thienyl, 3-thienyl, 3-methyl-2-thienyl, 4-bromo-2-thienyl, 5-bromo-2-thienyl, 5-nitro-2-thienyl, 5-methyl-2-thienyl, 5-(4-methoxyphenyl)-2-thienyl, 4-methyl-2-thienyl, 3-phenoxy-2-thienyl, 5-carboxy-2-thienyl, 2,5-dichloro-3-thienyl, 3-methoxy-2-thienyl, 2-benzothienyl, 3-methyl-2-benzothienyl, 2-bromo-5-chloro-3-benzothienyl, 2-thiazolyl, 2-amino-4-chloro-5-thiazolyl, 2,4-dichloro-5-thiazolyl, 2-diethylamino-5-thiazolyl, 3-methyl-4-nitro-5-isoxazolyl, 2-pyridyl, 3-pyridyl, 4-pyridyl, 6-methyl-2-pyridyl, 3-hydroxy-5-hydroxymethyl-2-methyl-4-pyridyl, 2,6-dichloro-4-pyridyl, 3-chloro-5-trifluoromethyl-2-pyridyl, 4,6-dimethyl-2-pyridyl, 4-(4-chlorophenyl)-3-pyridyl, 2-chloro-5-methoxycarbonyl-6-methyl-4-phenyl-3-pyridyl, 2-chloro-3-pyridyl, 6-(3-trifluoromethylphenoxy)-3-pyridyl, 2-(4-chlorophenoxy)-3-pyridyl, 2,4-dimethoxy-5-pyrimidine, 2-quinolinyl, 3-quinolinyl, 4-quinolinyl, 2-chloro-3-quinolinyl, 2-chloro-6-methoxy-3-quinolinyl, 8-hydroxy-2-quinolinyl and 4-isoquinolinyl,
or a salt thereof.

4. A compound of the formula 1 as claimed in claim 1, in which
R1 is hydrogen, a radical CH₂—R11, or a radical CH(OR12)-R11,
where
R11 is hydrogen, 1-4C-alkyl, aryl, tetrahydrofuryl, pyridyl, furyl or thienyl,
R12 is hydrogen, 1-4C-alkyl, 3-7C-cycloalkyl, 3-7C-cycloalkyl-1-4C-alkyl, 1-4C-alkoxy-1-4C-alkyl or fluoro-1-4C-alkyl,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
R2 is 1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, 1-4C-alkoxycarbonyl, cyanomethyl, carboxyl, or the radical —CO—NR31R32,
where
R31 is hydrogen, 1-7C-alkyl, hydroxy-1-4C-alkyl, 1-4C-alkoxy-1-4C-alkyl, or 3-7C-cycloalkyl, and
R32 is hydrogen or 1-7C-alkyl,
or where
R31 and R32 together and including the nitrogen atom to which they are attached form a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl or the radical —CO—NR41R42,
where
R41 is hydrogen, 1-7C-alkyl, hydroxy-1-4C-alkyl, 1-4C-alkoxy-1-4C-alkyl, or 3-7C-cycloalkyl and
R42 is hydrogen or 1-7C-alkyl,
or where
R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6,
where
R5 is hydrogen, 1-4C-alkyl, hydroxy-1-4C-alkyl, 1-4C-alkoxy, 1-4C-alkoxycarbonyl, halogen, trifluoromethyl, amino, mono- or di-1-4C-alkylamino, 1-4C-alkylcarbonylamino, 1-4C-alkoxycarbonylamino or 1-4C-alkoxy-1-4C-alkoxycarbonylamino and
R6 is hydrogen, 1-4C-alkyl or 1-4C-alkoxy,
or a salt thereof.

5. A compound of the formula 1 as claimed in claim 1, in which
R1 is hydrogen, a radical CH₂—R11, or a radical CH(OR12)-R11,
where
R11 is hydrogen, 1-4C-alkyl, aryl, tetrahydrofuryl, pyridyl, furyl or thienyl,
R12 is hydrogen, 1-4C-alkyl, 1-4C-alkoxy-1-4C-alkyl or fluoro-1-4C-alkyl,
where
aryl is phenyl or substituted phenyl having one substituent selected from the group consisting of 1-4C-alkyl, 1-4C-alkoxy, halogen, and hydroxyl,
R2 is 1-4C-alkyl,
R3 is hydrogen, 1-4C-alkyl, halogen, 2-4C-alkenyl, hydroxy-1-4C-alkyl, 1-4C-alkoxycarbonyl, carboxyl, or the radical —CO—NR31R32,
where
R31 is hydrogen, 1-7C-alkyl, hydroxy-1-4C-alkyl or 1-4C-alkoxy-1-4C-alkyl and
R32 is hydrogen or 1-7C-alkyl,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, or the radical —CO—NR41R42,
where
R41 is hydrogen, 1-7C-alkyl, hydroxy-1-4C-alkyl, 1-4C-alkoxy-1-4C-alkyl, or 3-7C-cycloalkyl and
R42 is hydrogen or 1-7C-alkyl,
or where
R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6,
where
R5 is hydrogen, 1-4C-alkyl or hydroxy-1-4C-alkyl and
R6 is 1-4C-alkyl,
or a salt thereof.

6. A compound of the formula 1 as claimed in claim 1, in which
R1 is hydrogen, a radical CH₂—R11, or a radical CH(OR12)-R11,
where
R11 is hydrogen, 1-4C-alkyl, phenyl, tetrahydrofuryl, furyl or thienyl,
R12 is hydrogen or 1-4C-alkyl,
R2 is 1-4C-alkyl,
R3 is hydrogen, 1-4C-alkyl, halogen, 2-4C-alkenyl or hydroxy-1-4C-alkyl,
R4 is carboxyl, 1-4-C-alkoxycarbonyl, halogen or the radical —CO—NR41R42,
where
R41 is hydrogen, 1-4C-alkyl, hydroxy-1-4C-alkyl or 3-7C-cycloalkyl and
R42 is hydrogen or 1-4C-alkyl,
or where
R41 and R42 together and including the nitrogen atom to which they are attached are a pyrrolidino, piperidino, morpholino, aziridino or azetidino radical, optionally substituted by a hydroxy radical,
Ar is a phenyl group substituted by R5 and R6,
where
R5 is hydrogen, 1-4C-alkyl or hydroxy-1-4C-alkyl and
R6 is 1-4C-alkyl,
or a salt thereof.

7. A compound of the formula 1 as claimed in claim 1, in which
R1 is hydrogen or a radical CH₂—R11,
where
R11 is hydrogen or phenyl,
R2 is 1-4C-alkyl,
R3 is hydrogen, 1-4C-alkyl, halogen or hydroxy-1-4C-alkyl,
R4 is carboxyl, 1-4C-alkoxycarbonyl, halogen or the radical —CO—NR41R42,
where
R41 is 1-4C-alkyl and
R42 is hydrogen or 1-4C-alkyl,
Ar is a phenyl group substituted by R5 and R6,
where
R5 is 1-4C-alkyl and
R6 is 1-4C-alkyl,
or a salt thereof.

8. A compound of the formula 1 as claimed in claim 1, in which
R1 is hydrogen,
R2 is 1-4C-alkyl,
R3 is hydrogen, halogen or hydroxy-1-4C-alkyl,
R4 is carboxyl, halogen or the radical —CO—NR41R42,
where
R41 is 1-4C-alkyl and
R42 is 1-4C-alkyl,
Ar is a phenyl group substituted by R5 and R6,
where
R5 is 1-4C-alkyl and
R6 is 1-4C-alkyl,
or a salt thereof.

9. A compound of the formula 1 as claimed in claim 1, characterized by the formula 1-1 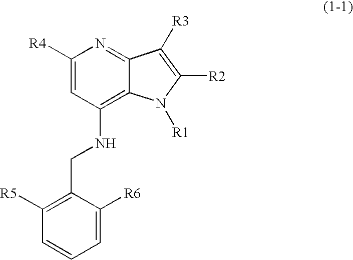
in which
R1 is hydrogen, a radical CH₂—R11 or a radical CH(OR12)-R11,
where
R11 is hydrogen, 1-4C-alkyl or a group Cy,
R12 is hydrogen, 1-4C-alkyl, 3-7C-cycloalkyl, 3-7C-cycloalkyl-1-4C-alkyl, 1-4C-alkoxy-1-4C-alkyl, 1-4C -alkoxycarbonyl, fluoro-1-4C-alkyl, 1-4C-alkylcarbonyl or the radical —CO—N(R121)(R122),
where
R121 is hydrogen, 1-7C-alkyl, hydroxy-1-4C-alkyl, 1-4C-alkoxy-1-4C-alkyl or 3-7C-cycloalkyl and
R122 is hydrogen or 1-7C-alkyl,
or where
R121 and R122 together and including the nitrogen atom to which they are attached form a pyrrolidino, piperidino, morpholino, aziridino or azetidino radical,
R2 is hydrogen, 1-4C-alkyl, 3-7C-cycloalkyl, 3-7C-cycloalkyl-1-4C-alkyl, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R3 is hydrogen, 1-4C-alkyl, 3-7C-cycloalkyl, 3-7C-cycloalkyl-1-4C-alkyl, halogen, 2-4C-alkenyl, 2-4C-alkynyl, hydroxy-1-4C-alkyl, aryl, hydroxy-3-4-C-alkenyl, hydroxy-3-4C-alkinyl, 1-4C-alkoxycarbonyl, fluoro-1-4C-alkyl, cyanomethyl, hydroxyl, 1-4C-alkoxy, amino, mono- or di-1-4C-alkylamino, 1-4C-alkylcarbonylamino, 1-4C-alkoxycarbonylamino, carboxyl, mono- or di-1-4C-alkylamino-1-4C-alkyl, 1-4C-alkylcarbonyl, 2-4C-alkenylcarbonyl, 2-4C-alkinylcarbonyl or the radical —CO—NR31R32,
where
R31 is hydrogen, 1-7C-alkyl, hydroxy-1-4C-alkyl, 1-4C-alkoxy-1-4C-alkyl or 3-7C-cycloalkyl and
R32 is hydrogen or 1-7C-alkyl,
or where
R31 and R32 together and including the nitrogen atom to which they are attached form a pyrrolidino, piperidino, morpholino, aziridino or azetidino radical,
R4 is hydroxy-1-4C-alkyl, 1-4C-alkoxy-1-4C-alkyl, 1-4C-alkoxy-1-4C-alkoxy-1-4C-alkyl, carboxyl, 1-4-C-alkoxycarbonyl, halogen, fluoro-1-4C-alkyl, 2-4C-alkenyl, 2-4C-alkynyl, cyano, or the radical —CO—NR41R42,
where
R41 is hydrogen, 1-7C-alkyl, hydroxy-1-4C-alkyl, 1-4C-alkoxy-1-4C-alkyl or 3-7C-cycloalkyl and
R42 is hydrogen or 1-7C-alkyl,
or where
R41 and R42 together and including the nitrogen atom to which they are attached are a pyrrolidino, piperidino, morpholino, aziridino or azetidino radical, optionally substituted by a hydroxy radical,
R5 is hydrogen, 1-4C-alkyl, hydroxy-1-4C-alkyl, 1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, aryl, aryl-1-4C-alkyl, aryl-oxy, aryl-1-4C-alkoxy, trifluoromethyl, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
R6 is hydrogen, 1-4C-alkyl, 1-4C-alkoxy, 1-4C-alkoxycarbonyl, halogen, trifluoromethyl or hydroxyl,
where
aryl is phenyl or substituted phenyl having one, two or three identical or different substituents selected from the group consisting of 1-4C-alkyl, 1-4C-alkoxy, carboxyl, 1-4C-alkoxycarbonyl, halogen, trifluoromethyl, nitro, trifluoromethoxy, hydroxyl and cyano,
and Cy is a tetrahydrofuryl group or a mono- or bicyclic aromatic residue as defined for Ar or aryl,
or a salt thereof.

10. A compound of the formula 1-1 as claimed in claim 9, in which
R1 is hydrogen or a radical CH₂—R11,
where
R11 is hydrogen or phenyl,
R2 is 1-4C-alkyl,
R3 is hydrogen, 1-4C-alkyl, halogen or hydroxy-1-4C-alkyl,
R4 is carboxyl, 1-4C-alkoxycarbonyl, halogen or the radical —CO—NR41R42,
where
R41 is 1-4C-alkyl and
R42 is hydrogen or 1-4C-alkyl,
R5 is 1-4C-alkyl and
R6 is 1-4C-alkyl,
or a salt thereof.

11. A compound of the formula 1-1 as claimed in claim 9, in which
R1 is hydrogen,
R2 is 1-4C-alkyl,
R3 is hydrogen, halogen or hydroxy-1-4C-alkyl,
R4 is carboxyl, halogen or the radical —CO—NR41R42,
where
R41 is 1-4C-alkyl and
R42 is 1-4C-alkyl,
R5 is 1-4C-alkyl and
R6 is 1-4C-alkyl,
or a salt thereof.

12. A pharmaceutical composition comprising a compound as claimed in claim 1 and/or a pharmacologically acceptable salt thereof together with a pharmacologically acceptable auxiliary and/or excipient.

13. A method of preventing or treating a gastrointestinal disorder in a patient comprising administering to a patient in need thereof a compound as claimed in claim 1 and/or a pharmacologically acceptable salt thereof.

14. (canceled)
